SEQUENCE LISTING (16 sequences)

SEQ ID NO: 1 - RNA, 21 nt, Artificial Sequence
  Description of Artificial Sequence Synthetic oligonucleotide
gagaugaacuaaaucgagcua

SEQ ID NO: 2 - DNA, 21 bp, Artificial Sequence
  Description of Combined DNA/RNA Molecule Synthetic oligonucleotide
  Description of Artificial Sequence Synthetic oligonucleotide
gaacaagcgaugcaugaagtt

SEQ ID NO: 3 - RNA, 21 nt, Artificial Sequence
  Description of Artificial Sequence Synthetic oligonucleotide
gacaucaaguaaaucgagcua

SEQ ID NO: 4 - DNA, 21 bp, Artificial Sequence
  Description of Combined DNA/RNA Molecule Synthetic oligonucleotide
  Description of Artificial Sequence Synthetic oligonucleotide
auuguaugcgaucgcagactt

SEQ ID NO: 5 - DNA, 21 bp, Artificial Sequence
  Description of Artificial Sequence Synthetic oligonucleotide
ggatgctacaattaaggtgtg

SEQ ID NO: 6 - DNA, 21 bp, Artificial Sequence
  Description of Artificial Sequence Synthetic oligonucleotide
aggctttaaccaagcagataa

SEQ ID NO: 7 - DNA, 28 bp, Artificial Sequence
  Description of Artificial Sequence Synthetic primer
ggaagatctccaggaattctgctgtccc

SEQ ID NO: 8 - DNA, 19 bp, Artificial Sequence
  Description of Artificial Sequence Synthetic primer
taaccgcggccgctcaacg

SEQ ID NO: 9 - DNA, 21 bp, Artificial Sequence
  Description of Artificial Sequence Synthetic primer
taaccgcggccgctactgacc

SEQ ID NO: 10 - DNA, 1233 bp, Homo sapiens
atggtgctgtcccagagacaacgagatgaactaaatcgagctatagcagattatcttcgttcaaatggctatgaagaggcatattcagtttttaaaaaggaagctgaattagatgtgaatgaagaattagataaaaagtatgctggtcttttggaaaaaaaatggacatctgttattagattacaaaagaaggttatggaattagaatcaaagctaaatgaagcaaaagaagaatttacgtcaggtggacctcttggtcagaaacgagacccaaaagaatggattccccgtccgccagaaaaatatgcattgagtggtcacaggagtccagtcactcgagtcattttccatcctgtgttcagtgttatggtctctgcttcagaggatgctacaattaaggtgtgggattatgagactggagattttgaacgaactcttaaaggacatacagactctgtacaggacatttcattcgaccacagcggcaagcttctggcttcctgttctgcagatatgaccattaaactatgggattttcagggctttgaatgcatcagaaccatgcacggccatgaccacaatgtttcttcagtagccatcatgcccaatggagatcatatagtgtctgcctcaagggataaaactataaaaatgtgggaagtgcaaactggctactgtgtgaagacattcacaggacacagagaatgggtacgtatggtacggccaaatcaagatggcactctgatagccagctgttccaatgaccagactgtgcgtgtatgggtcgtagcaacaaaggaatgcaaggctgagctccgagagcatgagcatgtggtagaatgcatttcctgggctccagaaagctcatattcctccatctctgaagcaacaggatctgagactaaaaaaagtggtaaacctgggccattcttgctgtctggatccagagacaagactattaagatgtgggatgtcagtactggcatgtgccttatgaccctcgtgggtcatgataactgggtacgtggagttctgttccattctggggggaagtttattttgagttgtgctgatgacaagaccctacgcgtatgggattacaagaacaagcgatgcatgaagaccctcaatgcgcatgaacactttgttacctccttggatttccacaagacggcaccctatgtcgtcactggcagcgtagatcaaacagtaaaagtgtgggagtgccgttga

SEQ ID NO: 11 - DNA, 13941 bp, Homo sapiens
atgtcggagcccgggggcggcggcggcgaggacggctcggccggattggaagtgtcggccgtgcagaatgtggcggacgtgtcggtgctgcagaagcacctgcgcaagctggtgccgctgctgctggaggacggcggcgaggcgccggccgcgctggaggcggcgctggaggagaagagcgccctggagcagatgcgcaagttcctttcggacccgcaggtccacacggtgctggtggagcgctccacgctcaaagaggacgtcggtgatgaaggagaagaagaaaaagaattcatttcctataacatcaacatagacattcattatggggttaaatccaatagcttggcattcattaaacgtactcccgtgattgatgcagataaacccgtgtcttctcagctccgggtccttacactcagtgaagactcgccctacgaaactttgcattctttcattagcaatgcagtggctcctttttttaagtcctacattagagagtctggcaaggcagacagggatggtgataaaatggctccttcagttgaaaagaagattgcagaactcgaaatgggactccttcacttgcagcaaaatattgaaattccggagatcagcctgccgattcatccaatgatcacaaatgttgcaaaacagtgttatgagcgtggagaaaagccaaaagttacagactttggtgataaggttgaagacccaacatttcttaatcagttacaatctggagttaaccgctggatccgagaaattcaaaaagtgaccaaactggatcgagatcctgcatcaggaactgccttacaggaaattagtttttggctaaacttggaacgtgcgttataccgcatccaggagaaacgggagagcccggaagttctcctgactctggatatcttgaaacatggcaagcgcttccatgccaccgtcagttttgacactgacacaggtctaaaacaggctttggaaactgtgaatgactacaatcctctgatgaaagatttccctctgaatgatttgctgtctgccacggagctggacaaaataagacaggcgcttgttgccattttcacacatttgagaaagatccgaaacacaaaatatcctattcagagggcactgcgtttggtggaggcaatttcaagagacttgagttctcaattactcaaagtattgggcactaggaaattgatgcatgttgcttatgaagaatttgaaaaagttatggtagcatgctttgaagtttttcagacttgggatgatgagtatgagaaacttcaggtattgttgagagacatcgtcaaaagaaaaagggaagaaaatctgaagatggtgtggcgtatcaaccctgcccacaggaagctgcaggcccgccttgaccagatgagaaaatttagacgccagcatgaacagctaagagctgttatcgtcagggtcctgaggccacaggtcacggcagttgcacaacagaatcaaggagaggtccctgaaccccaagatatgaaagtggctgaggttctctttgatgctgcagatgcaaatgccattgaggaagtaaaccttgcttatgagaacgtcaaggaagtggatggactggatgtttccaaagagggcacggaagcctgggaggctgctatgaagaggtacgatgagaggatcgacagagtggagacccggatcaccgctcgccttcgggatcagcttggcacagccaagaatgccaacgagatgtttaggattttctccaggtttaatgcactgtttgtcaggcctcacatccgtggggccattcgcgaataccagacccagctgatccagcgcgtgaaagatgacattgagtctcttcacgacaagttcaaggtccagtacccacagagtcaggcttgtaagatgagtcacgttcgtgacttgccccctgtgtcagggtctatcatctgggctaaacagatcgacaggcagctgacggcctacatgaagcgggtggaagatgtccttggcaagggctgggagaatcacgtggaggggcagaagctgaagcaggatggagacagcttccgcatgaagctcaacacgcaggagatctttgatgactgggcaaggaaggtgcagcagcgcaacctcggtgtctcggggcgcattttcaccatcgaaagtactcgggttcggggccgaactggaaatgtgcttaagctgaaagttaactttcttcctgagattatcacactatccaaagaagtccggaacctcaaatggcttggtttccgcgtcccactggcgattgtgaacaaagcccatcaagcaaaccagctttacccgtttgccatctcactgatcgagagcgttcgtacctatgaacggacctgcgagaaggtggaggagcggaacaccatttcccttttggtggctggcttgaaaaaggaagtgcaggccctgatcgcagaaggcattgcgttggtgtgggagtcctacaaacttgacccatatgtacagcgcttagcagagactgtcttcaacttccaagaaaaggtggatgatctgctgatcattgaagaaaaaatagacctagaagtccgttccttggaaacttgtatgtatgaccataagacattctcggaaatcttgaacagagtccagaaagcagtggatgacttaaatctgcactcctattccaatttgcccatctgggtcaacaagcttgacatggagattgaaagaatattgggcgtccgtctgcaagctggcctgagagcttggacgcaggttcttcttggacaagctgaagataaagcagaagttgacatggacacagatgctccacaagttagtcacaagcctggtggagagccaaagatcaaaaatgtcgttcatgagctaagaataaccaatcaggtaatctacttgaatccaccaattgaagagtgcagatacaagctgtatcaggaaatgtttgcctggaagatggttgtactgtctctccccaggatccagagtcagaggtaccaggtgggtgtacattacgaattgactgaggaagagaaattctatcggaatgctttaacacggatgcctgatggccctgttgccctggaagagtcgtattctgctgtcatgggcattgtatctgaagttgaacagtatgtcaaggtttggcttcagtatcagtgtttatgggatatgcaagctgaaaacatctataacagacttggagaagatctcaacaaatggcaggctctcctggtccaaataaggaaggccagaggaacctttgacaatgcagaaaccaagaaagagtttggaccagtagttatagattatggcaaggtacaatctaaggtgaacttgaaatatgactcttggcataaggaggttcttagcaaatttgggcagatgctaggatcaaacatgacggaattccattcccagatctcaaagtcccgccaagagttggagcagcactcagtagacacggccagcacctccgatgcagtgaccttcatcacctatgtgcagtctttgaaacggaagatcaagcagtttgagaagcaagttgagctctaccgcaatggccagcgcttactggaaaagcaaaggttccagttcccaccttcctggctttatattgacaacatcgagggagagtggggagccttcaatgacatcatgcggcgaaaggactctgccattcagcagcaggtggcaaacctgcaaatgaagattgtccaggaggatcgggccgtggaaagccgcaccaccgacctgctgactgactgggagaagaccaagcctgtcacgggcaaccttcgcccagaagaggcacttcaggctctcaccatatatgaggggaagtttggtaggctgaaggacgacagagagaagtgtgcaaaggccaaggaggcgctggaattgacagatactgggcttctcagtggcagtgaagagcgcgtgcaggtggccttagaagaattacaggacctcaaaggcgtttggtcagaactttctaaggtttgggagcaaatcgatcagatgaaggagcaaccctgggtttcagtacagcctcgaaagcttcgacaaaatttggatgccctcctgaaccagctgaaaagcttccctgcccggttgcgacagtatgcgtcctatgagtttgttcagaggcttctgaaaggttacatgaagataaatatgctggtgattgaactgaaatccgaagcacttaaagaccgccattggaaacagctcatgaaaaggcttcacgttaattgggttgtttctgagctaacccttggccaaatctgggatgttgacttgcagaaaaatgaagcgattgtcaaggatgtactgcttgtggcacaaggggagatggctttggaagaatttttgaagcagataagagaagtgtggaatacttatgaactagacttggttaattatcagaacaagtgccgcttgatccgtggctgggatgacctcttcaacaaggtcaaagaacacatcaacagcgtctcggccatgaagctctctccgtattacaaggtttttgaagaggatgctctcagctgggaagataagctgaacaggatcatggctctctttgatgtgtggattgatgtgcagaggcggtgggtctacctggaaggtatcttcacaggcagtgcagatatcaagcacctgctgccagtggaaacccagcggtttcagagcatcagcactgagtttttggctctaatgaaaaaagtgtccaagtctccccttgttatggatgttctgaacatccagggagtacagaggtctctggaaagattggcagacctgctaggaaagatccagaaagcattgggagaatatctggaaagagagcggtcatctttccccaggttctattttgtgggtgatgaagatttgcttgaaatcattggaaacagcaagaatgtcgctaaattacagaaacacttcaagaagatgtttgctggagtttcgagcatcatcctgaacgaggataactctgttgttttgggtatttcatctcgggaaggagaggaggttatgtttaaaactcctgtgtcaattactgaacatcccaaaatcaatgagtggctcacattggtagaaaaggagatgagagtcaccctggccaaactgcttgctgagtctgttacggaagttgagatttttggtaaagcaacttcaattgacccaaatacctacatcacttggattgataaataccaggcccagcttgtggttttgtcagcccagatagcctggtctgagaacgtggagaccgcactgagcagcatgggcggaggtggagatgccgcgcccttgcactctgtgctgagcaatgtggaggtcaccctcaatgtgttagcagactctgtcctcatggagcagcccccactccgaaggcggaagctagaacacttgattacagagttggttcaccagagagatgttacaaggtccttgatcaaaagcaagattgacaacgccaaatcttttgaatggctcagccagatgcgattttactttgaccctaagcaaactgatgtgttacagcagttgtcaattcaaatggcaaatgccaaatttaactatggctttgagtacctgggtgttcaggacaaactggtccagacccccctcactgaccgctgctatttgacaatgacacaagccttggaggccaggctggggggttccccatttggacctgctggaactgggaaaacagagtctgtcaaagctcttggccatcagcttggacggtttgttttagttttcaactgtgatgaaacctttgatttccaggcaatgggccggatctttgtgggcctttgccaggtgggtgcctggggctgctttgacgagttcaaccgcctggaggagcggatgctctcggctgtgtcccagcaggtgcagtgcatacaggaagcactgcgtgaacattccaaccccaactacgacaagacctctgcccccattacttgtgagctgctgaacaaacaagtcaaggtgagcccggacatggccatcttcatcaccatgaaccctggctacgcgggccggtctaaccttcctgacaacttgaagaagctgttccggagcttggccatgaccaagcccgaccggcagttaatcgcccaggtcatgctgtactcacagggtttccgcactgctgaagtgcttgccaacaaaatcgtcccgttttttaaactatgcgatgagcagctctcttcccaaagccattatgacttcggtcttcgggctttgaagagtgtgctggtgagtgcaggcaatgtgaagagagagagaatccagaagataaagagggagaaagaggaacgaggggaagcagttgatgaaggagaaattgctgaaaatctccctgaacaagagattctgatacagagcgtctgtgagacgatggtgccaaagctggtggcagaggacatcccgctgctcttcagcctcctgtcggacgtgttccctggagtccagtatcacaggggtgagatgactgcccttcgagaggagctgaagaaagtgtgtcaggagatgtatttgacatatggagatggagaagaagttggtggaatgtgggttgaaaaggttctccagctctatcagatcacccagatcaatcatggcctgatgatggtggggccctcgggaagtgggaagagcatggcctggcgtgtcctgctgaaggcattggagagactcgagggtgtggaaggtgtggcccatatcatcgaccccaaggccatcagcaaagaccacctctacggaaccctggaccccaacaccagggaatggacagatgggctcttcacacacgtgctgagaaagatcatcgacagcgtgagaggcgagctgcagaagcgccagtggatcgtcttcgatggcgatgtggatccagagtgggttgagaacttgaactcagtgctggatgacaataagctcctaactttgcccaatggagagcgcctcagtcttccacccaatgtgagaataatgtttgaggtacaggacttgaaatacgcgaccttggccacagtgtcgcgctgcggcatggtctggttcagtgaggatgtgctgagcaccgacatgatcttcaacaacttcctggccaggctgcgcagcatcccgctggatgaaggggaggatgaggcacagcggcggcgtaagggcaaagaggatgagggggaggaggccgcttcccccatgctgcagatccaaagagatgcagctacgatcatgcaaccgtacttcacgtccaacggcctggtcaccaaggcgctagagcacgccttccagctggagcacatcatggacctaacacgcctgcgctgcctgggctcgctcttctccatgctgcaccaggcctgccgcaacgtggcgcagtataacgccaaccatcccgacttccccatgcagatcgagcagctggagcgctacattcagcgatatctggtttatgccatactctggtccctgtctggagacagccggctaaaaatgagagcagagctgggtgaatacatcagaagaatcacgaccgtgcctctgcccactgcgcccaacatacccattatcgattatgaggtgtccatcagcggagaatggtctccgtggcaggccaaggtgcctcagattgaagtggagacgcacaaggtggcagcccctgatgtcgtcgtgccaacgctggacacagtccgccacgaagccctcttgtacacttggctggccgaacacaagcccctggtcttgtgtggccctcctgggtctggcaagaccatgacactcttcagcgccctccgggccttgcctgacatggaggtggtgggtctcaacttctccagtgctactactccagagctgcttctgaagacttttgatcactactgcgagtacaggcgcacacctaatggggtggttttggctcctgttcaacttggaaagtggctggtgttgttctgtgatgaaatcaacttgccagatatggataaatatgggacccagagggtcatatccttcatcagacagatggtggagcacggaggcttttaccgtacctcagatcaaacatgggtgaagctggagagaatccagtttgttggggcttgtaatccccccacagaccctggaagaaagcccctctcacacaggttcctgcgccacgtgcctgtcgtgtatgtggattacccgggccccgcctccctcacacagatctacggcaccttcaaccgcgccatgctgaggctcattccatccctgcggacgtatgcagagccgctcactgctgccatggtggagttctacaccatgtctcaggagagattcacccaggatacacaacctcactatatctattcaccccgtgaaatgactaggtgggtgagaggcatctttgaagcgctgagacctctggagaccctgcctgttgaaggcctcattcggatttgggcacatgaagctctgcgtctcttccaagatagactcgtagaggatgaggagaggcgttggactgatgagaacatcgacacggttgctctgaagcacttccctaacatcgacagagagaaggcaatgagccgacccatcttgtacagcaactggctgtcaaaggattacatcccagtagaccaagaagagttaagagattatgtcaaagctaggctgaaggtcttttatgaagaagaacttgatgttccgctggtgctgtttaatgaagtcctagaccacgtgctgaggattgacagaatattccgtcaacctcaaggccacttgcttctgattggtgttagtggagcaggaaaaactaccctgtctcgtttcgtcgcctggatgaacggtttgagtgtgtaccagattaaggtccataggaagtacacaggggaagactttgatgaagatctacggacagtgttgagacgttctggctgtaaaaatgaaaagatagcatttataatggatgaatctaatgtgttagattctggattcctggagcgaatgaatacccttctggccaatggagaggtgcctggtctctttgaaggagacgagtatgccaccttgatgacgcagtgcaaagagggggcacagaaggaaggcctgatgctggactcgcacgaggagctctacaagtggttcactagccaggttatccgcaacctccacgtcgtgttcaccatgaacccgtcctcggagggactcaaggaccgggcagctacatcaccagcacttttcaacaggtgtgtgttgaattggtttggagactggtccaccgaagcactgtatcaggttggcaaagaattcacaagtaagatggatctggagaagccaaattacatcgtgcctgattacatgccagttgtgtatgataagctgccgcagccaccatcccatcgggaagccattgtgaacagctgtgtgtttgttcatcagactcttcaccaggcgaatgctcggctagcaaagcgaggcggcagaacgatggccatcacccctcgccactacctggacttcatcaatcactatgccaacctgttccacgagaagcggagcgagctggaggagcagcagatgcacttgaacgtggggctcaggaagatcaaagagacagtcgaccaggtagaagaactgcgtcgtgacttgaggataaagagccaagagctggaggtgaagaatgcagcagccaatgacaagctgaaaaagatggtgaaagaccagcaggaggctgaaaagaagaaggttatgagccaagaaatccaggaacagctgcataagcagcaggaggtaattgcagacaaacagatgagtgtcaaagaagatcttgataaggtggaacctgccgtcattgaggcccagaatgctgtgaagtcgatcaagaagcagcacctggtggaggtgaggtccatggccaaccctcctgctgctgtgaagctggcgctggagtccatctgcctgctgctgggggaaagcaccacagactggaagcagatccgctccatcatcatgcgggagaacttcatccccaccatcgtcaacttctctgcagaggagatcagtgacgccataagggagaagatgaagaaaaattacatgtccaatccaagttacaattatgaaattgtgaatcgggcttccctggcttgcggccctatggtgaaatgggcaattgcacagcttaactatgcagacatgttaaagagagtggagcccctacgcaatgagctgcagaagctggaagatgacgccaaggacaaccagcagaaggccaacgaggtggagcagatgatccgagacctggaagccagcatcgcccgctacaaggaggaatacgccgtcctgatctcagaggcccaggccatcaaggcagacctggcagctgtcgaggcaaaagtaaaccggagcactgctcttctgaagagcttgtctgctgaacgtgaacgatgggaaaaaacaagtgaaactttcaaaaaccagatgtccaccattgctggggactgtctcttgtcagctgcgttcattgcctacgcgggttactttgaccagcagatgcgtcagaacttgttcactacctggtcccatcacctacagcaagccaacatccagttccgtacagatattgccaggacggaatacctttccaatgctgatgagcgtcttcgctggcaggccagctccttgcctgctgatgacctttgcacagaaaatgccatcatgctgaaacgattcaataggtatccgctgatcattgacccctctggacaggccacagaattcattatgaatgaatataaggatcgtaagatcacacggaccagcttcctggatgacgccttcagaaagaacttagagagtgcactgagattcggtaacccccttctggtccaggatgtggaaagctacgatccagttttgaacccggtgctgaaccgtgaagtgcggcgaacaggggggagagtgctgatcactctcggggaccaggacatagacctgtcgccatcgtttgtcatcttcctgtccacccgggatccaactgtcgagttcccaccagatctctgttcccgggttacttttgtaaacttcacagttacccgtagcagtttacaaagccagtgtctaaatgaagtacttaaagcagaaagacctgatgtggacgagaaacgatctgatcttcttaaacttcaaggggaatttcagctccgtttgcgtcagctggaaaaatctctactacaagctctgaacgaggtgaaagggcgcattttggatgacgacacgatcataaccactctggagaacctgaagagagaggctgcagaggtcaccaggaaagttgaggagacggacattgtcatgcaggaggtggagaccgtgtcccagcagtacctcccgctctccaccgcctgcagcagcatctacttcaccatggagtccctcaagcagatacacttcttgtaccagtactccctccagtttttcctggacatttatcacaacgtcctatacgagaacccgaacctgaagggtgtcaccgaccacacacagcgcctgtccattataacaaaggacctcttccaggtggcgtttaaccgagtggctcgaggcatgctgcatcaggaccacattacctttgccatgctgctggcaagaatcaaactgaagggcaccgtgggggagcccacctacgatgcagaattccagcacttcttgagaggaaatgagattgtcctgagtgctggctccacccccaggatccagggcctgactgtggagcaggcggaggcggtggtgaggctgagctgccttcccgcgtttaaggacttgattgcaaaggttcaggcagacgagcaatttggcatctggctggacagcagctccccggagcagactgtgccctacctctggagtgaagaaacacctgcaacacccattggccaggccatccaccgcctgctcctgatccaggctttccggcccgatcgcctgttggccatggcccacatgtttgtttcaacaaaccttggggagtctttcatgtccatcatggagcagccgctcgacctgacccacattgtgggcacagaggtgaagcccaacactcctgtcttaatgtgctctgtgcctggttatgatgccagtggacatgtcgaggaccttgcagccgagcagaacacgcagatcacttcaattgcaatcggctctgcagaaggctttaaccaagcagataaggcaataaacaccgctgtaaagtcgggcaggtgggtgatgctgaagaatgtgcatctggccccagggtggctgatgcagctggagaagaagttgcattccctgcagccgcatgcctgcttccgactcttcctcaccatggagatcaaccccaaggtgcctgtgaatctgctccgtgcgggccgcatctttgtgttcgagccaccgccaggggtgaaggccaacatgctgaggacgttcagcagcattcccgtctcacggatatgcaagtctcccaacgagcgtgcccgcttgtacttcctgctggcctggtttcatgcgatcatccaagaacgcttacgatacgcaccactggggtggtcaaagaagtatgaatttggagagtctgacctgcggtcagcttgcgatacggtggacacgtggctggatgacacggccaagggcaggcagaacatctcaccggataagatcccgtggtctgcactaaagaccttaatggcccagtccatttatggcgggcgcgtggacaacgagtttgaccagcgtctgctcaacaccttcctggagcgcctgttcacaaccaggagtttcgacagtgagtttaagctggcatgcaaggtcgacggacataaagacattcaaatgccagatggcatcaggcgagaggagtttgtgcagtgggtggagttgctccccgacacccagacgccctcctggctgggcctgcccaacaacgccgagagagtcctccttaccacacagggtgtggacatgatcagtaaaatgctgaagatgcagatgttggaggatgaggacgacctggcctacgcagagactgagaagaagacgaggacagactccacgtccgacgggcgccctgcctggatgcggacactgcacaccaccgcgtccaactggctgcacctcatcccccagacgctgagccacctcaagcgcaccgtggagaatatcaaggatcctttgttcaggttctttgagagagaagtgaagatgggcgcaaagctgcttcaggacgttcgccaggaccttgcagatgtcgtccaggtgtgcgaaggaaagaagaagcagaccaactacttgcgcacgctgatcaacgagctagtgaaagggatcttgcctcggagctggtcccactacacggtgcctgccggcatgaccgtcatccagtgggtgtccgacttcagcgagaggatcaaacagctgcagaacatctcactggcagctgcatctggtggcgccaaggagctaaagaacatccacgtgtgcctgggtggcctgttcgtgcctgaggcgtacatcactgccaccaggcagtatgtggcccaggccaacagctggtccctggaggagctctgcctggaagtcaacgtcaccacctcacagggcgccacccttgacgcttgcagcttcggagtcacgggtttgaaacttcaaggggccacgtgcaacaacaacaagctgtcactgtccaatgccatctcaaccgcccttcccctgacgcagctgcgctgggtcaagcagacaaacaccgagaagaaggccagtgtggtaaccttacctgtctacctgaacttcacccgtgcagacctcatcttcaccgtggacttcgaaattgctacaaaggaggatcctcgcagcttctacgagcggggtgtcgcagtcttgtgcacagagtaa

SEQ ID NO: 12 - DNA, 1221 bp, Homo sapiens
atggcggaccctaaatacgccgaccttcccggcattgccaggaatgagccagatgtttatgaaactagcgacctacctgaggatgatcaagcggagttcgatgcgtttgcacaagagctggaggagctgacaagcacaagtgtggaacacatcattgtcaatcctaatgctgcctatgacaagttcaaggacaagagagtggggacaaagggacttgatttctcagatcgtattggaaaaaccaagaggacaggatatgaatctggagaatatgagatgcttggagagggtctgggagtgaaggagacaccccagcaaaagtaccagcgcctactgcatgaggtccaagagctgacaactgaagttgaaaaaatcaagacgacagtgaaggagtcagccacagaggagaagctgacccctgtgttgctggctaaacagctggcagccctgaagcagcagctggttgcttcccacctggagaagctgctgggaccagatgctgcaatcaaccttaccgaccccgatggcgccctggctaagcgcctactactgcagctggaagcaacaaagaacagcaaagggggatcagggggaaaaaccactgggacccccccagatagcagccttgtcacttatgaactacattctcggcctgagcaggacaagttctctcaagctgccaaagtcgcagaacttgaaaagcgcctgacagagctggagacagctgtacgttgtgatcaggatgctcagaatcccctttctgcaggtctacagggagcctgtctcatggagactgtagagctgttgcaagcaaaggtgagcgccctagaccttgcagttttggatcaagtggaggctcggctacagagtgtcctgggaaaggtgaacgagattgccaagcataaagcctctgtagaagatgcagatacacaaagcaaggtgcaccagctatatgaaactatacagcgctggagccccattgcctccaccctccctgagctggtgcagagacttgtcaccatcaagcagctgcacgagcaagccatgcagtttggtcagctcctgacacacttggataccacccagcagatgattgctaattccttgaaggacaataccaccctcttgacccaggtgcagacaaccatgcgtgaaaacctggccacagttgaggggaactttgccagcattgatgaacggatgaagaagctgggaaagtga

SEQ ID NO: 13 - protein, 410 aa, Homo sapiens
MVLSQRQRDELNRAIADYLRSNGYEEAYSVFKKEAELDVNEELDKKYAGLLEKKWTSVIRLQKKVMELESKLNEAKEEFTSGGPLGQKRDPKEWIPRPPEKYALSGHRSPVTRVIFHPVFSVMVSASEDATIKVWDYETGDFERTLKGHTDSVQDISFDHSGKLLASCSADMTIKLWDFQGFECIRTMHGHDHNVSSVAIMPNGDHIVSASRDKTIKMWEVQTGYCVKTFTGHREWVRMVRPNQDGTLIASCSNDQTVRVWVVATKECKAELREHEHVVECISWAPESSYSSISEATGSETKKSGKPGPFLLSGSRDKTIKMWDVSTGMCLMTLVGHDNWVRGVLFHSGGKFILSCADDKTLRVWDYKNKRCMKTLNAHEHFVTSLDFHKTAPYVVTGSVDQTVKVWECR

SEQ ID NO: 14 - protein, 87 aa, Homo sapiens
MVLSQRQRDELNRAIADYLRSNGYEEAYSVFKKEAELDVNEELDKKYAGLLEKKWTSVIRLQKKVMELESKLNEAKEEFTSGGPLGQ

SEQ ID NO: 15 - protein, 323 aa, Homo sapiens
KRDPKEWIPRPPEKYALSGHRSPVTRVIFHPVFSVMVSASEDATIKVWDYETGDFERTLKGHTDSVQDISFDHSGKLLASCSADMTIKLWDFQGFECIRTMHGHDHNVSSVAIMPNGDHIVSASRDKTIKMWEVQTGYCVKTFTGHREWVRMVRPNQDGTLIASCSNDQTVRVWVVATKECKAELREHEHVVECISWAPESSYSSISEATGSETKKSGKPGPFLLSGSRDKTIKMWDVSTGMCLMTLVGHDNWVRGVLFHSGGKFILSCADDKTLRVWDYKNKRCMKTLNAHEHFVTSLDFHKTAPYVVTGSVDQTVKVWECR

SEQ ID NO: 16 - protein, 406 aa, Homo sapiens
MADPKYADLPGIARNEPDVYETSDLPEDDQAEFDAFAQELEELTSTSVEHIIVNPNAAYDKFKDKRVGTKGLDFSDRIGKTKRTGYESGEYEMLGEGLGVKETPQQKYQRLLHEVQELTTEVEKIKTTVKESATEEKLTPVLLAKQLAALKQQLVASHLEKLLGPDAAINLTDPDGALAKRLLLQLEATKNSKGGSGGKTTGTPPDSSLVTYELHSRPEQDKFSQAAKVAELEKRLTELETAVRCDQDAQNPLSAGLQGACLMETVELLQAKVSALDLAVLDQVEARLQSVLGKVNEIAKHKASVEDADTQSKVHQLYETIQRWSPIASTLPELVQRLVTIKQLHEQAMQFGQLLTHLDTTQQMIANSLKDNTTLLTQVQTTMRENLATVEGNFASIDERMKKLGK

(19) United States
(12) Patent Application Publication
(10) Pub. No.: US 2007/0213286 A1
(43) Pub. Date: Sep. 13, 2007

(54) Inhibitors of dynein or lissencephaly 1, and methods of using same for treatment of neuronal disorders

(75) Inventors: Richard B. Vallee, Dobbs Ferry, NY (US); Peter Canoll, New York, NY (US); Jin-Wu Tsai, New York, NY (US); James Goldman, White Plains, NY (US)

Correspondence Address: WilmerHale/Columbia University, 399 PARK AVENUE, NEW YORK, NY, 10022, US

(73) Assignee: The Trustees of Columbia University in the City of New York, New York, NY (US)

(21) Appl. No.: 11/516,204

(22) Filed: Sep. 6, 2006

Related U.S. Application Data
(60) Provisional application No. 60/716,210, filed on Sep. 12, 2005.

(51) Int. Cl. A61K 48/00; C07H 21/02

(52) U.S. Cl. 514/44; 536/23.1

(57) ABSTRACT

The invention provides antagonists and agonists of dynein mediated activity, for example specific inhibitory RNAs and dominant negative cDNAs which function as antagonists of LIS1 and dynein, and compositions comprising such antagonist and agonists. Provided are also methods for treating unwanted cell proliferation of neural progenitor cells such as cancers of the CNS, including gliomas, by administering the antagonists of the invention.

This application claims the benefit of U.S. Provisional Application Ser. No. 60/716,210, filed on Sep. 12, 2005, the contents of which are hereby incorporated by reference in its entirety.

The invention disclosed herein was made with U.S. Government support under NIH Grant Nos. NS15429-25 and HD40182. Accordingly, the U.S. Government has certain rights in this invention

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

### BACKGROUND

Gliomas (primary brain tumors) start in the brain or spinal cord. Gliomas typically do not spread to other areas of the body but can spread within the nervous system. Gliomas can be either benign, slow growing, or malignant, fast growing. Grading references how tumor cells look under the microscope. Grades 1 and 2 are low grade, Grade 3 is moderate and Grade 4 is high. Low grade means that the tumor cells resemble normal brain cells; they usually grow slowly and are not likely to spread. In high grade tumors, the cells look very abnormal, and are more likely to grow quickly and spread.

Types of gliomas include: astrocytomas, ependymomas, oligodendrogliomas. The most common gliomas, astrocytomas, start in brain cells called astrocytes and can occur in most parts of the brain, and occasionally in the spinal cord. However, they are most commonly found in the main part of the brain, the cerebrum. People of all ages can develop astrocytomas, but they are more common in adults, particularly middle-aged men. Astrocytomas in the base of the brain are more common in children or young people. Different astrocytomas include: low grade astrocytomas, which may occur in the cerebrum in adults and children or in the cerebellum of children; anaplastic astrocytomas which are mid-grade tumors which commonly spread to surrounding brain tissue; glioblastoma multiforme—grade 4 astrocytomas, which are also the most malignant. Glioblastoma multiforme usually spread quickly to other parts of the brain. For this reason, these are difficult to treat. It is not uncommon for these tumors to recur after the initial treatment, and further treatment may be needed.

Ependymomas are tumors which begin in the ependyma, the cells that line the passageways in the brain where special fluid that protects the brain and spinal cord (called cerebrospinal fluid) is made and stored. They are a rare glioma and can be found anywhere in the brain or spine, but most commonly in the main part of the brain, the cerebrum. Ependymomas may spread from the brain to the spinal cord via the cerebrospinal fluid.

Oligodendrogliomas are primary brain tumors which begin in the brain cells called oligodendrocytes, which provide support and nourishment for the cells that transmit nerve impulses. This tumor is normally found in the cerebrum, the main part of the brain.

Mixed gliomas are brain tumors of more than one type of brain cell, including cells of astrocytes, ependymal cells and/or oligodendrocytes. The most common site for a mixed glioma is the cerebrum, the main part of the brain. Like other gliomas, they may spread to other parts of the brain.

Malignant gliomas are notoriously difficult to treat. Standard treatment is by surgery to reduce the tumor size, followed by radiotherapy therapy which remains the main post-surgical treatment. Adding chemotherapy to the treatment results in a small but significant prolongation of survival. Despite aggressive treatment of these cancers, there has been little improvement in survival of patients. Thus there is need for improved therapeutic methods and composition to treat gliomas.

### SUMMARY OF THE INVENTION

In certain aspects, the invention provides an isolated nucleic acid that encodes an inhibitory RNA (iRNA) that inhibits Lissencephaly1 (LIS1) function. In one aspect, the nucleic acid that encodes iRNA, which inhibits LIS1 function, can comprise a nucleic acid sequence derived from SEQ ID NO: 10. In other aspects, the iRNA sequence can be selected from, but is not limited to sequences selected from the group consisting of: SEQ ID NO: 1, SEQ ID NO: 2, and SEQ ID NO: 5. In other aspects, the invention provides an isolated nucleic acid that encodes an inhibitory RNA molecule that inhibits dynein function. In one aspect, the nucleic acid that encodes iRNA, which inhibits dynein function, can comprise a nucleic acid sequence derived from SEQ ID NO: 11. In a certain aspect, the iRNA sequence is SEQ ID NO: 6. In certain aspects, the invention provides an isolated nucleic acid that encodes an inhibitory RNA molecule that inhibits dynactin function. In other aspects, the nucleic acid that encodes iRNA, which inhibits dynactin function, can comprise a nucleic acid sequence derived from SEQ ID NO: 12.

In certain aspects, the invention provides an isolated nucleic acid that encodes an inhibitory RNA that inhibits Lissencephaly1 (LIS1) function, the nucleic acid comprising a nucleic acid sequence as listed in SEQ ID NO: 1, SEQ ID NO: 2, or SEQ ID NO: 5. In certain aspects, the nucleic acid sequence which encodes iRNA is about 95%, 96%, 97%, 98%, 99%, 99.9% identical to any one of SEQ ID NOS: 1, 2, 5, or 6. In other aspects, the nucleic acid sequence which encodes iRNA comprises an addition, substitution, deletion or insertion of one or more nucleotides compared to any one of SEQ ID NOS: 1, 2, 5 or 6.

In other aspects, the invention provides a method for treating a disease or disorder associated with abnormal growth or migration of neural progenitor cells, the method comprising administering to a subject an effective amount of a nucleic acid that encodes an inhibitory RNA that inhibits LIS1, dynein or dynactin function. In other aspects, the invention provides a method for treating a glioma, the method comprising administering to a subject an effective amount of a nucleic acid that encodes an inhibitory RNA that inhibits LIS1, dynein or dynactin function. In other aspects, the invention provides a method for treating a disease or disorder associated with abnormal growth or migration of neural progenitor cells, the method comprising administering to a subject an effective amount of a nucleic acid that encodes a dominant negative polypeptide that inhibits LIS1, dynein or dynactin function. In other aspects, the invention provides a method for treating a glioma, the method comprising administering to a subject an effective amount of a nucleic acid that encodes a dominant negative polypeptide that inhibits LIS1, dynein or dynactin function. In certain aspects, the disease or disorder is a cancer of the central nervous system. In other aspects, the disease or disorder is glioma, meningioma, medulloblastoma, neuroectodermal tumor, epyndymoma, or any combination thereof. In certain aspects of the methods, the abnormal growth and/or migration is undesirable. In certain aspects of the methods, the nucleic acid is administered to the disease or disorder site. In certain aspects of the methods, the nucleic acid is administered to the cancer site. In certain aspects of the methods, the nucleic acid is administered intracranially.

In certain aspects, the nucleic acid sequence of the inhibitory RNA can be of various length, such as from about 15 to about 35 nucleotides. In other aspects, the nucleic acid sequence of the inhibitory RNA comprises a sense and anti-sense strand which can form an RNA duplex. In certain embodiments, the sense and antisense strands are covalently linked by a single-stranded hairpin. In certain aspects, the nucleic acid sequence of the inhibitory RNA comprises non-nucleotide molecule. In certain aspects, the nucleic acid sequence of the inhibitory RNA can be about 90%, 91%, 92%, 93%, 94%, 95%, 96%, 97%, 98%, 99% homologous, or identical, to any particular target gene sequence. In certain aspects, the nucleic acid sequence of the inhibitory RNA comprises an addition, substitution, deletion or insertion of one or more nucleotides compared to any one to any particular target gene sequence.

In other aspects, the invention provides a dominant negative polypeptide or an isolated nucleic acid, or a cDNA that encodes dominant negative polypeptide that inhibits the function of LIS1. In other aspects, the invention provides a dominant negative polypeptide or an isolated nucleic acid, or a cDNA that encodes dominant negative polypeptide that inhibits the function of dynein. In other aspects, the invention provides a dominant negative polypeptide or an isolated nucleic acid, or a cDNA that encodes dominant negative polypeptide that inhibits the function of dynactin. In certain aspects, the invention provides an isolated polypeptide that inhibits Lissencephaly1 (LIS1) function, the polypeptide comprising the amino acid sequence as listed in SEQ ID NO: 13, 14 or 15. In other aspects, the invention provides an isolated polypeptide that inhibits dynactin function, the polypeptide comprising the amino acid sequence as listed in SEQ ID NO: 16. In certain embodiments, dominant negative polypeptide of the invention can comprise any modification such as additions, deletions, and/or substitutions.

In certain aspects, the invention is directed to an isolated nucleic acid that encodes inhibitory RNA or dominant negative cDNA molecules that inhibit the function of LIS1. In other aspects, the invention is directed to an isolated nucleic acid that encodes inhibitory RNA or dominant negative cDNA molecules that inhibit the function of dynein. In other aspects, the invention is directed to an isolated nucleic acid that encodes inhibitory RNA or dominant negative cDNA molecules that inhibit the function of dynactin. In other aspects, the invention is directed to composition comprising at least one or more of any one of a number of nucleic acids that encode inhibitory RNA or dominant negative cDNA molecules that inhibit the function of LIS1, dynein, dynactin, or LIS1-, dynein-, dynactin-mediated activity.

In other aspect, the invention provides a composition comprising at least one agent selected form among: at least one nucleic acid of encoding iRNA or dominant negative molecule, or a dominant negative molecule as provided herein. In certain embodiments, the composition comprises pharmaceutical material. In certain aspects, a nucleic acid of the invention can be comprises in an expression vector. In certain aspects, the expression vector can be an expression plasmid, recombinant plasmid, or viral vector. In other aspects, a nucleic acid of the invention can be comprised in a pharmaceutical composition.

In other aspects, the invention provides an expression vector that comprises a nucleic acid of encoding iRNA or dominant negative molecule as provided herein. In certain embodiments, the expression vector is selected from but not limited to the group consisting of: expression plasmid, recombinant plasmid, and viral vector. In other aspects, the invention provides a composition comprising an expression vector as provided herein.

In other aspects, the invention provides a method for treating a disorder associated with abnormal or unwanted growth, proliferation, division, morphogenesis or migration of neural progenitor cells, wherein the method comprises administering to a subject an effective amount of any of the nucleic acids of the invention encoding inhibitory RNA, dominant negative molecule, or any antagonist of LIS1, dynein, dynactin, or LIS1-, dynein-, dynactin-mediated activity. In other aspects, the invention provides a method for treating a disorder associated with abnormal or unwanted growth, proliferation, division, morphogenesis or migration of neural progenitor cells, wherein the method comprises administering to a subject an effective amount of a dominant negative molecule inhibiting LIS1, dynein, dynactin, or LIS1-, dynein-, dynactin-mediated activity. In certain aspects, the disorder associated with abnormal or unwanted growth, proliferation, division, morphogenesis or migration of neural progenitor cells is a cancer or tumor of the central nervous system including glioma, meningioma, medulloblastoma, neuroectodermal tumor, epyndymoma, or any combination thereof.

In other aspects, the invention provides a method for treating a glioma, or inhibiting dynein mediated activity in a subject, the method comprising administering to a subject suffering from a glioma an effective amount of any of the nucleic acids of the invention encoding inhibitory RNA, dominant negative molecule, or any other antagonist of LIS1, dynein, dynactin, or LIS1-, dynein-, dynactin-mediated activity.

In other aspects, the invention provides a method for inhibiting cell division of neural progenitor cells, wherein the method comprises contacting a neural progenitor cell with an effective amount of any of the nucleic acids of the invention encoding inhibitory RNA, dominant negative molecule, or any other antagonist of LIS1, dynein, dynactin, or LIS1-, dynein-, dynactin-mediated activity.

In other aspects, the invention provides a method for inhibiting cell proliferation of neural progenitor cells, wherein the method comprises contacting a neural progenitor cell with an effective amount of any of the nucleic acids of the invention encoding inhibitory RNA, dominant negative molecule, or any other antagonist of LIS1, dynein, dynactin, or LIS1-, dynein-, dynactin-mediated activity.

In other aspects, the invention provides a method for inhibiting morphogenesis of neural progenitor cells, wherein the method comprises contacting a neural progenitor cell with an effective amount of any of the nucleic acids of the invention encoding inhibitory RNA, dominant negative molecule, or any other antagonist of LIS1, dynein, dynactin, or LIS1-, dynein-, dynactin-mediated activity.

In another aspect, the invention provides a method for inhibiting migration of neural progenitor cells, wherein the method comprises contacting a neural progenitor cell with an effective amount of any of the nucleic acids of the invention encoding inhibitory RNA, dominant negative molecule, or any other antagonist of LIS1, dynein, dynactin, or LIS1-, dynein-, dynactin-mediated activity.

In other aspects, the invention provides a method to inhibit cortical histogenesis, wherein the method comprises contacting a neural progenitor cell with an effective amount of any of the nucleic acids of the invention encoding inhibitory RNA, dominant negative molecule, or any other antagonist of LIS1, dynein, dynactin, or LIS1-, dynein-, dynactin-mediated activity.

In certain aspects, the methods of the invention inhibit or treat cell division, cell proliferation, morphogenesis, migration, or cortical histogenesis which are undesirable. In certain aspects, the effective amount of any of the nucleic acids of the invention encoding inhibitory RNA, dominant negative molecule, or any other antagonist of LIS1, dynein, dynactin, or LIS1-, dynein-, dynactin-mediated activity, is administered directly to the site of undesirable cell division, cell proliferation, morphogenesis, migration, or cortical histogenesis of neural progenitor cells. In certain embodiments of the methods, the effective amount of any of the nucleic acids of the invention is administered directly to the tumor site, regardless whether the tumor is still present or has been treated, and removed. In certain embodiment, the effective amount of any of the nucleic acids of the invention is administered directly the site of a glioma, before or after the glioma has been removed. In certain embodiments of the methods, the effective amount of any of the nucleic acids of the invention is administered intracranially.

In other aspects, the invention provides methods for inhibiting cell proliferation of neural progenitor cells, wherein the method comprises contacting a nucleic acid of the invention to a neural progenitor cell. In other aspects, the invention provides methods for inhibiting morphogenesis of neural progenitor cells, wherein the method comprises contacting a nucleic acid of the invention to a neural progenitor cell. In other aspects, the invention provides methods for inhibiting migration of neural progenitor cells, wherein the method comprises contacting a nucleic acid of the invention to a neural progenitor cell. In other aspects, the invention provides methods for treating disorders associated with abnormal growth or migration of neural progenitor cells, wherein the method comprises administering to a subject an effective amount of any of the nucleic acids of the invention. In certain aspects, the methods of treating disorders associated with abnormal growth is directed to treating a disorder such as a glioma.

In certain aspects, the invention provides methods for identifying an agent that inhibits the function of LIS1, dynactin or dynein, wherein the method comprises: a) contacting a cell, for example but not limited to a neural progenitor cell with an agent, b) determining whether the cell exhibits reduced LIS1, dynactin, or dynein function, wherein reduced LIS1, dynactin or dynein function is indicative of an agent that inhibits the function of LIS1, dynactin or dynein. In certain aspect, the invention provides a method for identifying an agent that inhibits LIS1 function, wherein the method comprises: a) contacting a cell with an agent, b) determining whether the cell exhibits reduced LIS1 function, wherein reduced LIS1 function is indicative of an agent that inhibits the function of LIS1. In certain aspect, the invention provides a method for identifying an agent that inhibits dynein function, wherein the method comprises: a) contacting a cell with an agent, b) determining whether the cell exhibits reduced dynein function, wherein reduced dynein function is indicative of an agent that inhibits the function of dynein. In certain aspect, the invention provides a method for identifying an agent that inhibits dynactin function, wherein the method comprises: a) contacting a cell with an agent, b) determining whether the cell exhibits reduced dynactin function, wherein reduced dynactin function is indicative of an agent that inhibits the function of dynactin.

In certain embodiments, the invention provides methods for identifying agents that inhibit the function of LIS1, dynactin or dynein, wherein the determining step comprises comparing levels of cell proliferation, morphogenesis or cell migration by the cell in the presence of an agent with the levels determined in the absence of the agent. In certain the cell used in the methods for identifying an agent can be, but is not limited to, neural progenitor cell, or cell of neural lineage, or a cell derived from a cancer of neural lineage, for example but not limited to a glioma. In certain aspects of the methods for identifying an agent, the determining step comprises comparing levels of cell proliferation, morphogenesis or cell migration by the cell in the presence of the agent with the levels determined in the absence of the agent.

In other aspects, the invention provides methods for treating or preventing or ameliorating lissencephaly in a subject, the method comprising administering to the brain of the subject an effective amount of LIS1, dynein and/or dynactin, as a nucleic acid encoding the stated peptides or portions thereof, so as to increase expression of LIS1, dynein and/or dynactin and thereby treating or preventing or ameliorating the lissencephaly.

In other aspects, the invention provides methods to inhibit cortical histogenesis so as to treat a disorder or disease associated with cortical histogenesis, the method comprising administering to a brain, or the cortex of a subject an effective amount of an inhibitor of LIS1, dynein and/or dynactin, for example but not limited to, siRNA, RNAi, and the like. In certain aspects, administering is directly to the subject's brain.

In other aspects, the invention provides methods to treat a disorder or abnormality of cortical histogenesis comprising administering to a subject, for example, specifically to the brain of the subject an agent that increases the expression or presence of LIS1, such as LIS1 itself, or a nucleic acid encoding LIS1, dynein, such as dynein or a nucleic acid encoding dynein, and/or of dynactin, such as dynactin itself, or a nucleic acid encoding dynactin, so as to improve cortical histogenesis and progression of neural progenitors through the cell cycle in the ventricular zone. In certain aspects, administering is directly to the subject's brain.

In other aspects, the invention provides methods for increasing neuronal progenitor proliferation, division and/or migration comprising administering to a subject, for example, specifically to the brain of the subject an agent that increases the expression or presence of LIS1, such as LIS1 peptide itself, or a nucleic acid encoding LIS1, dynein, such as dynein or a nucleic acid encoding dynein, and/or of dynactin, such as dynactin itself, or a nucleic acid encoding dynactin so as to increase neuronal progenitor proliferation, division and/or migration in the subject. In certain aspects, administering is directly to the subject's brain.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 shows effect of RNAi on LIS1 expression and neural progenitor cell distribution. (A, top) Western blot of LIS1 and tubulin in COS7 cells 48 h after transfection with LIS1 shRNA vectors or oligonucleotides. LIS1 levels in cells transfected with LIS1 shRNA or oligonucleotides were much lower than those in cells transfected with triple point mutated shRNA, empty vector, or control oligonucleotide. (Bottom) Immunostaining of LIS1 (left panel) in E18 rat neocortical neural progenitor cells transfected in utero at E16 with LIS1 shRNA vector, which also expresses GFP marker (right panel). Note the loss of LIS1 in GFP-expressing cells (arrows). (B) Disruption of cell redistribution in the neocortex by LIS1 RNAi. Coronal sections of rat brain 2, 4, and 6 d after electroporation at E16 with LIS1 shRNA, control shRNA, or empty vector. Cells expressing LIS1 shRNA were largely restricted to the VZ/SVZ, although some appeared within the lower IZ by days 4 and 6 (left). In contrast, cells transfected with control shRNA or empty vector migrated radially from the VZ to the CP with increasing time (middle and right). Note the additional lateral spread of VZ/SVZ cells in the control. Arrows, axonlike processes extending from migratory bipolar cells (see Altered Axonal Extension). Bar, 100 μm. (C) Percentages of cells transfected with empty, control shRNA, LIS1 shRNA vectors, and LIS1 siRNA oligonucleotide in different regions of the neocortex. The signal of Cy3-siRNA-transfected cells at day 6 was too low to detect. Error bars represent SEM. t test: *, P<0.05; **, P<0.01.

FIG. 2 shows morphology of LIS1 shRNA-transfected cells. (A) Morphology of cells transfected with control (top) and LIS1 shRNA (bottom) vectors (green) 2, 4, and 6 d after electroporation at E16 and counterstained with antinestin antibody (red). Many control cells migrated to the IZ and CP, where they became bipolar (arrows), whereas most LIS1 shRNA-transfected cells remained in the SVZ with a multipolar morphology (arrowheads). Bar, 50 μm. (B) Quantitative effects of LIS1 RNAi on cell morphology. In control brains, GFP-expressing radial glial and multipolar cells were maximal at posttransfection day 2 and decreased with time, and the number of bipolar cells increased. In LIS1 shRNA- and siRNA-transfected brains, the numbers of radial glial and multipolar cells remained relatively constant with time. (C) Comparison of bipolar cell number under diverse inhibitory conditions 2 d after electroporation. All dominant negative and siRNA constructs markedly decreased the ratio of bipolar to total transfected cells. Error bars represent SEM. t test: *, P<0.05.

FIG. 3 shows cell cycle stage of transfected cells. Brains were transfected with empty vector, LIS1 shRNA, or GFP-LIS1N (green) at E16 and, 2 d later, were immunostained with antibodies to the M-phase marker phosphovimentin (4A4, red; top), and Ki67 (red; bottom), which is a transcription factor expressed from S through M phase. Transfected cells positive for either marker appear yellow (arrows). Bar, 50 μm. The percentage of total transfected cells that were positive for 4A4 or Ki67 was reduced significantly in LIS1 shRNA- and GFP-LIS1N-transfected brains relative to controls. Error bars represent SEM. t test: *, P<0.05; **, P<0.01.

FIG. 4 shows live cell imaging of the conversion of neural progenitor cells from multipolar to bipolar morphology. (A) Slices from rat brain electroporated in utero with control or LIS1 shRNA vector at E16 were placed into culture at E18 and imaged every 10 min by GFP fluorescence microscopy. Multiple processes of the control neuron at the top (arrows) give way to a single major process at the top as the cell initiates radial migration. Cell expressing shRNA for LIS1 (bottom) persisted in the multipolar state for 18 h of observation. Time is shown in hours/minutes. Bar, 10 μm. (B) Number of primary processes emanating from a multipolar cell body varied over a broad but similar range in control versus LIS1 shRNA-transfected cells. (C) Number of process branch points per cell were increased in LIS1 siRNA-transfected cells versus controls.

FIG. 5 shows live cell imaging of neural progenitor cell behavior within the VZ. (A) Cell body of a control progenitor cell at the radial glial stage migrates away from and then toward the ventricular surface (dotted lines), where it divides by the last time point. Tracings of cell body positions for five typical controls show rapid but discontinuous movements. Arrows indicate the time of cell division. (B) Cell body of a LIS1 shRNA-transfected cell is relatively immobile over a 14-h time period. Tracings show that the cell body position is relatively constant and that stepwise changes are not observed. Note that the behavior of cell bodies in control and experimental cases are independent of starting position relative to the ventricular surface. Times are shown in hours/minutes. Bar, 5 μm.

FIG. 6 shows live cell imaging of neural progenitor cell behavior within the IZ. Rat brains were electroporated with LIS1 (bottom) or control shRNA (top) constructs at E16, and the brains were sectioned and cultured 3 d later. (A) Images from bipolar cells within the IZ. Control cells extended a leading process toward the CP, and the cell body followed, resulting in forward locomotion with a process of relatively constant length. When transfected with LIS1 shRNA, the leading process of the cells continued to grow, but the cell body remained immobile. The leading process also extended many short projections along its length. Time is shown in hours/minutes. Bar, 5 μm. (B) Branching of leading process in the IZ. Control cells normally had one to three branches, whereas there were many small branches in LIS1 shRNA-transfected cells. (C) Rate of leading process extension and somal migration. The rate of process extension was almost unchanged by LIS1 RNAi, but somal movement was largely abolished. Error bars represent SEM. t test: **, P<0.01.

FIG. 7 shows live cell imaging of axonlike processes. Rat brain was electroporated with control or LIS1 RNAi construct at E16. (A) Fixed images of axonlike processes in control (vector) cells 4 d posttransfection (top). These processes extend tangentially toward the medial line from most postmitotic neurons in the SVZ and IZ. In LIS1 shRNA-transfected brains, axonlike processes were observed in the same orientation, although they were shorter, more curved, and more branched (bottom panels show days 4 and 6 posttransfection). (B) Most of the axonlike processes in LIS1 shRNA-transfected cells could be seen to originate from multipolar cells. Bar, 100 μm. Box shows the magnification of a multipolar cell extending an axonlike process. Bar, 20 μm. (C) Live cell imaging of axonal growth. Rat brain sections were placed into culture 2 d after electroporation, and images were recorded from the SVZ every 10 min. (Left) The control axons usually possessed only one branch point (arrowheads), with the two branches alternatively growing (yellow arrows) and retracting (blue arrows), resulting in an overall steady pattern of axonal elongation (0.7 μm/min in example shown). (Right) Axons transfected with LIS1 shRNA construct exhibited multiple short branches (arrowheads indicate branch points), which extended and retracted dynamically. The overall length of the axon, however, stayed virtually constant. Time is shown in hours/minutes. Bar, 10 μm. (D) Axonal growth rate. Axons of LIS1 shRNA-transfected cells grew much more slowly than controls. (E) Axonal branching was also substantially increased in LIS1 shRNA-transfected cells.

FIG. 8 is a schematic diagram showing the effects of LIS1 inhibition on neural progenitor cell proliferation, migration, and morphogenesis. (Top) Cortical neurons undergo distinct phases of neurogenesis and migration (Noctor et al., 2004). (a) The first step involves bidirectional interkinetic nuclear oscillations within radial glial cells (light green/gray). Cell division occurs at the ventricular surface. (b) Postmitotic neurons (dark green/gray) then migrate away from the ventricle and become paused in a multipolar state within the VZ/SVZ as they extend an axon. (c) Cells convert to a bipolar morphology and migrate along radial fibers to the CP as axonal growth continues. (d) As the orientation of radial fibers becomes distorted by cortical expansion, neuronal migration becomes increasingly outward directed. (Bottom) Neural progenitor cells exhibit as many as three discrete terminal effects of LIS1 RNAi. (a′) Radial glial cells cease interkinetic nuclear migration, and cell division is inhibited. (b′) Multipolar cells fail to transition to the bipolar migratory stage. (c′) Bipolar cells extend a leading process at normal rates, but the cell soma remains stationary. Although axons are extended from both multipolar and bipolar cells, the axons are more curved, branched, and grow more slowly. (d′) A preponderance of cells are arrested within the SVZ, resulting in subcortical band heterotopia, which is associated with classical lissencephaly. Both radial migration and lateral dispersion are disrupted, preventing neuronal cells from inserting into the CP and from contributing to further lateral spread. Blue and orange arrows indicate cell body movements and process extension, respectively. Red crosses indicate blocking of these activities.

FIG. 9 shows that neuronal migration occurs radially but also along curved radial glial fiber tracks. Cellular routes for neocortical expansion. (A) Coronal section of rat brain at E20 immunostained for radial glial fibers using antinestin antibody (red). Neuronal cells were marked (green) by transfecting GFP cDNA and using in utero electroporation at E16. As noted in altered distribution inhibition, neurons migrated along straight radial tracks as well as along tracks that have been distorted laterally. Radial glial fibers exhibit a similar distribution. Bar, 200 μm. (B) Migrating neurons in the IZ. A magnification from A (boxed area) showing the leading processes of migrating neurons aligning with the radial fibers. Bar, 100 μm. (C) An alignment of leading processes with radial fibers is shown that used vimentin (red) as a glial marker. Bar, 50 μm.

FIG. 10 shows that multipolar morphology is predominant in neural progenitor cells that were transfected with Cy3-LIS1 siRNA oligonucleotides or dominant negative LIS1 and dynamitin constructs. Morphology of cells with altered LIS1 or dynein function. Cells were electroporated in utero with Cy3-LIS1 siRNA (red and double arrowheads; cotransfected with GFP in green), GFP-LIS1N, and dynamitin (GFP-p50, green) and were fixed at day 2. Most of the cells exhibited a multipolar morphology (arrowheads) similar to that seen in pRNAT-LIS1 brains. Bar, 50 μm.

FIG. 11 shows that multipolar cells expressing LIS1 shRNA are positive for the neuronal marker TuJ1. Neuronal identity of LIS1 shRNA-transfected cells. Brains were transfected with pRNAT-LIS1 (green) and, 2 days later, were immunostained with antibodies to the neuronal marker TuJ1 (red). (A) Most of the cells were positive to TuJ1 in the cell soma. Bar, 50 μm. (B) Enlargement of a multipolar cell from the boxed area in A. TuJ1 can be detected in the tiny cell soma around the nucleus. Bar, 20 μm.

FIG. 12 shows nucleic acid sequence encoding human LIS1 (SEQ ID NO: 10).

FIG. 13 shows nucleic acid sequence encoding cytoplasmic human dynein heavy chain (SEQ ID NO: 11).

FIG. 14 shows nucleic acid sequence encoding human dynamitin (p50) (SEQ ID NO: 12).

FIG. 15 shows amino acid sequence of human LIS1 (SEQ ID NO: 13).

FIG. 16 shows amino acid sequence of the N-terminus of human LIS1, amino acid position 1 to 87 (SEQ ID NO: 14).

FIG. 17 shows amino acid sequence of the C-terminus of human LIS1, amino acid position 88 to 410 (SEQ ID NO: 15).

FIG. 18 shows amino acid sequence of human dynamitin (SEQ ID NO: 16).

### DETAILED DESCRIPTION

Definitions

The terms “LIS1 antagonist” and “antagonist of LIS1” are used interchangeably herein to designate an agent that inhibits the activity of LIS1, or any agent that inhibits a positive regulator(s) of LIS1 activity, or LIS1 mediated activity. The terms “dynein antagonist” and “antagonist of dynein” are used interchangeably herein to designate an agent that inhibits the activity of dynein, or any agent that inhibits a positive regulator(s) of dynein activity, or dynein mediated activity. The terms “dynactin antagonist” and “antagonist of dynactin” are used interchangeably herein to designate an agent that inhibits the activity of dynactin, or any agent that inhibits a positive regulator(s) of dynactin activity, or dynactin mediated activity. An antagonist can be any of: small molecules, antibodies, polynucleotide compounds, such as but not limited to antisense polynucleotides, ribozymes, RNA interference molecules, triple helix polynucleotides and the like, where the nucleotide sequence of such compounds are related to the nucleotide sequences of DNA and/or RNA of LIS1, dynein, dynactin or any of their positive regulators. The term “inhibits” as used herein encompasses any reduction in levels of mRNA, protein, or activity of LIS1, dynein, and dynactin, or any of their positive regulators, or LIS1, dynein, dynactin mediated activity thereof.

LIS1, dynein, dynactin, any one of their positive regulators, and/or mediators of their function are also referred to as “target gene” or “target protein”.

A subject as used herein refers to either a human or a non-human animal subject suffering from a disorder associated with uncontrolled cell growth, cell division or proliferation, and/or migration of neural progenitor cells.

The terms “treatment” or “treat” as used herein include treating, preventing, ameliorating, and/or decreasing the severity of the symptoms of a disease or disorder, or improving prognosis for recovery.

The term “cortical histogenesis” as used herein comprises progression of neural progenitors through the cell cycle in the ventricular zone.

Mutations in the human LIS1 gene cause the smooth brain disease classical lissencephaly. The human developmental disease classical lissencephaly is characterized by a nearly complete absence of gyri, an abnormally thickened four-layered cortex, and enlarged ventricles (Dobyns and Truwit, 1995). Classical, or type I, lissencephaly results from sporadic mutations in the LIS1 gene, resulting in LIS1 haploinsufficiency (Reiner et al., 1993; Lo Nigro et al., 1997). LIS1 was initially identified as a noncatalytic subunit of platelet-activating factor acetylhydrolase Ib (Hattori et al., 1994). Deletion of the catalytic subunits of platelet-activating factor acetylhydrolase, however, affects testicular rather than brain development (Koizumi et al., 2003; Yan et al., 2003). LIS1 orthologues have also been identified in the cytoplasmic dynein pathway in lower eukaryotes (Xiang et al., 1995), and biochemical and molecular studies have shown that LIS1 interacts with cytoplasmic dynein and its regulatory complex dynactin (Faulkner et al., 2000; Sasaki et al., 2000; Smith et al., 2000). LIS1 also interacts with other proteins initially identified in the dynein pathway: NudC (Morris et al., 1998), NudE (Efimov and Morris, 2000; Kitagawa et al., 2000), and its isoform NudEL (Niethammer et al., 2000).

LIS1 orthologues are essential for nuclear migration and nuclear orientation in fungi (Xiang et al., 1995; Geiser et al., 1997). In dividing vertebrate cultured cells, LIS1 associates with kinetochores and the cell cortex (Faulkner et al., 2000). LIS1 also associates with centrosomes (Smith et al., 2000; Tanaka et al., 2004) and is located at the leading edge of migrating fibroblasts (Dujardin et al., 2003). Interference with LIS1 produces severe mitotic defects (Faulkner et al., 2000; Tai et al., 2002) and inhibits the redistribution of cerebellar granule cell soma within reaggregate cultures (Hirotsune et al., 1998) as well as the directed migration of fibroblasts (Dujardin et al., 2003; Kholmanskikh et al., 2003). How these cellular defects may contribute to the neuronal migration disorder and the agyric or pachygyric morphology of the lissencephalic brain is not completely understood.

Developmental analysis of LIS1 heterozygous mouse lines and of cells transfected with cDNA constructs for LIS1 RNA interference (RNAi) has shown abnormalities in the extent of neuronal redistribution (Hirotsune et al., 1998; Gambello et al., 2003; Shu et al., 2004). However, detailed analysis of the migration pathway has not been performed. Cortical neurons are generated directly from radial glial cells in the ventricular zone (VZ) or indirectly from intermediate progenitor cells in the subventricular zone (SVZ) that are themselves generated from radial glia (Noctor et al., 2001, 2004; Haubensak et al., 2004). Neural progenitor cells progress through a series of morphogenetic stages. After classic interkinetic nuclear oscillations and cell division at the ventricular surface, newborn neurons ascend to the SVZ, where they convert to a multipolar nonmigratory phase (Rakic et al., 1974; Tabata and Nakajima, 2003; Noctor et al., 2004). After about a day, during which they begin to extend axonal processes, they convert to a bipolar stage and resume glial-directed radial migration. The importance of this complex progression in cortical development, how it is regulated, and how defects in this pathway may contribute to developmental diseases such as lissencephaly is not yet well understood.

To gain insight into the specific role of LIS1 in neural progenitor behavior and neuronal cell migration, in situ live cell imaging analysis of neural progenitor cells with reduced LIS1 expression was conducted; and the behavior of these cells was followed throughout the migratory pathway. Migration and morphogenesis were blocked at multiple distinct stages, each of which has important implications for the biological function of LIS1 and for the physiological mechanisms underlying normal neurogenesis.

Human mutations in LIS1 cause the serious brain developmental disease lissencephaly resulting from decreased cerebral surface area. In humans and in rodent models, reduction in LIS1 protein levels interferes with neuronal migration and laminar organization. Using in utero electroporation, LIS1 siRNAs and dominant negative cDNAs were introduced into neural progenitor cells in embryonic rat neocortex. Radial migration was dramatically disrupted, and the transfected cells were stalled in the subventricular zone (SVZ), forming a heterotopic lamina of green fluorescent cells. The cells exhibited a multipolar morphology, indicating that LIS1 is required for progression from the recently identified multipolar to the bipolar phase, and that this transition is vital for neuronal migration. Cells overexpressing dominant negative dynactin revealed a similar phenotype, providing the direct evidence that LIS1 functions in brain development through the dynein pathway. The transfected cells extended aberrant branched axon-like processes, which grew slower than control cells, suggesting a novel function for LIS1 in axonal growth. Immunostaining using cell cycle markers revealed a decrease in S-phase and M-phase cells in the SVZ, which may be caused by reduced SVZ proliferation, mitotic cell death, or premature differentiation. A significant lateral migratory pathway along dramatically curved radial glial fibers was identified. This pathway was also disrupted by LIS1 inhibition, resulting in failure in the lateral dispersion of cells. These results demonstrate an important role for LIS1 and dynactin in progression from the multipolar to the bipolar migratory stage and a new model for cortical expansion in the rodent, which occurs not only by radial migration, but also by lateral dispersion.

To understand the underlying mechanisms of various defects caused by mutations in LIS1, in situ live cell imaging analysis of LIS1 function throughout the entire radial migration pathway was conducted. In utero electroporation of LIS1 small interference RNA and short hairpin dominant negative LIS1 and dynactin cDNAs caused a dramatic accumulation of multipolar progenitor cells within the subventricular zone of embryonic rat brains. This effect resulted from a complete failure in progression from the multipolar to the migratory bipolar state, as revealed by time-lapse analysis of brain slices. Interkinetic nuclear oscillations in the radial glial progenitors were also abolished, as were cell divisions at the ventricular surface. Those few bipolar cells that reached the intermediate zone also exhibited a complete block in somal translocation, although, remarkably, process extension persisted. Finally, axonal growth also ceased. These results identify multiple distinct and novel roles for LIS1 in nucleokinesis and process dynamics and suggest that nuclear position controls neural progenitor cell division.

In certain aspects, the invention provides that decrease or loss of LIS1 functions, and/or mediated activity results in defects and failure of cell cycle progression, cell proliferation of neural progenitor cells. Decrease or loss of LIS1 functions, and/or mediated activity results in defects in neuronal process outgrowth which seemed to block the passage of post mitotic precursors through the subventricular zone (SVZ) onto the radial glial pathway. In other aspects, decrease or loss of LIS1 functions, and/or mediated activity results in the accumulation of multipolar progenitor cells within the SVZ. In other aspects, decrease or loss of LIS1 functions, and/or mediated activity results in defects in interkinetic nuclear oscillation of radial glial progenitors.

LIS1 binds to vertebrate cytoplasmic dynein as demonstrated by several assays (Faulkner et al. 2000; Sasaki et al. 2000; Smith et al. 2000; Tai et al. 2002). The interaction is substoichiometric, and occurs through multiple sites. LIS1 interacts with three distinct sites within dynein itself, including the base of the molecule responsible for cargo binding, and the motor domain (Tai et al. 2002). LIS1 also interacts with the dynamitin subunit of dynactin, a dynein accessory complex which, itself, interacts with dynein and mediates its attachment to diverse subcellular structures. Aspergillus NudF also interacts with the NudE (Efimov and Morris 2000) and NudC genes, and vertebrate LIS1 physically associates with two NudE orthologues, mNudE (Kitagawa et al. 2000) and its isoform NudEL(Niethammer et al. 2000), and with mNudC (Morris et al. 1998). Each of these proteins, in turn, interacts with cytoplasmic dynein (Sasaki et al. 2000; Aumais et al. 2001). Additional LIS1/dynein or dynactin interactors, such as CLIP-170 (Coquelle and al. 2001; Komarova et al. 2002; Tai et al. 2002), have been identified, as have numerous binding partners of NudE and NudEL (Feng et al. 2000; Morris et al. 2003; Toyo-oka et al. 2003; Liang et al. 2004).

The physiological need for this degree of complexity is poorly understood. Cytoplasmic dynein, itself, is a mechanochemical enzyme, and NudEL as well as LIS1 associates with the dynein motor domain. It may be that the complexity in dynein composition and interaction partners relates to the fine control of dynein activity. In addition, cytoplasmic dynein participates in numerous cellular activities associated at diverse sites within the cell. Some functions involve transport of membranous organelles or macromolecule complexes, but others appear to involve tension generation. This range of roles may also require a highly complex range of regulatory factors and mechanisms.

In certain aspects, the invention provides that antagonists of LIS1 (lissencephaly type I), or antagonist to positive regulators if LIS1, lead to accumulation of multipolar neural progenitor cells within the subventricular zone of the brain. In other aspects, the invention provides that antagonists of LIS1, or antagonist to positive regulators if LIS1, lead to failure in progression from the multipolar to the migratory bipolar state of neural progenitor cells, including but not limited to radial glial progenitor cells. In other aspects, the invention provides that antagonists of LIS1, or antagonist to positive regulators if LIS1, abolish and/or lead to defects in interkinetic nuclear oscillations in neural progenitor cells, including but not limited to radial glial progenitors. In other aspects, the invention provides that antagonists of LIS1, or antagonist to positive regulators if LIS1, abolish and/or lead to cell division defects in neural progenitor cells, including but not limited to radial glial progenitors.

In other aspects, the invention provides that brain developmental disorders arising from mutations in the dynein pathway can be due to defects in cell proliferation as well as in migration.

Compositions:

In certain aspects, the invention provides antagonists to dynein mediated pathway, including LIS1, dynactin, and/or dynein. In other aspects, the present invention provides iRNAs which are antagonists to the dynein mediated pathway. In certain aspects, the present invention provides isolated nucleic acid sequences which encode molecules that interfere with the molecular function dynein or the dynein regulator LIS1 (lissencephaly type I). The molecules that interfere with LIS1, dynactin or dynein function can be iRNAs, such as vector based shRNA molecules, dsRNA molecules, or cDNA molecules that inhibit or reduce the function of LIS1 or dynein. Because inhibition of LIS1 affects division and migration, but not axonal transport, interference with LIS1 function may only have relatively mild effects on mature brain cells.

RNAi Antagonists: Antisense nucleotide technology has been a described approach in protocols to achieve gene-specific interference. For antisense strategies, stoichiometric amounts of single-stranded nucleic acid complementary to the messenger RNA for the gene of interest can be introduced into the cell. Triple helical nucleic acid structures are also useful for engineered interference. This approach relies on the rare ability of certain nucleic acid populations to adopt a triple-stranded structure. Under physiological conditions, nucleic acids are virtually all single- or double-stranded, and rarely if ever form triple-stranded structures.

In certain embodiments, an RNA interference (RNAi) molecule is used to decrease or inhibit expression of the nucleic acid against which the RNAi is directed. RNAi refers to the use of interfering RNA (iRNA) molecules for example but not limited to double-stranded RNA (dsRNA) or small interfering RNA (siRNA) to suppress the expression of a gene comprising a related nucleotide sequence. RNAi is also referred to as post-transcriptional gene silencing (or PTGS). The sequences inhibitory RNAs are based on the sequence of the target gene, and methods to design iRNAs are known in the art.

RNAi regulates gene expression via a ubiquitous mechanism by degradation of target mRNA in a sequence-specific manner. McManus et al., 2002, Nat Rev Genet 3:737-747. In mammalian cells, interfering RNA (RNAi) can be triggered by 21- to 23-nucleotide duplexes of siRNA. Lee et al., 2002, Nat Biotechnol 20: 500-505; Paul et al., 2002, Nat. Biotechnol. 20:505-508; Miyagishi et al., 2002, Nat. Biotechnol. 20:497-500; Paddison et al., 2002, Genes Dev. 16: 948-958. The expression of siRNA or short hairpin RNA (shRNA) driven by U6 promoter effectively mediates target mRNA degradation in mammalian cells.

Double-stranded (ds) RNA can be used to interfere with gene expression in many organisms including, but not limited to mammals. dsRNA is used as inhibitory RNA or RNAi of the function of a nucleic acid molecule of the invention to produce a phenotype that is similar to the phenotype of a mutant with decreased expression level and/or activity, for example but not limited to the phenotype of a null mutant of the target.

Many methods have been developed to make siRNA, e.g., chemical synthesis or in vitro transcription. Once made, the siRNA can be introduced directly into a cell to mediate RNA interference (Elbashir et al., 2001, Nature 411: 494-498; Song, E, et al. RNA interference targeting Fas protects mice from fulminant hepatitis. Nat. Med. 2003; 9: 347-351; and Lewis, D L, et al. Efficient delivery of siRNA for inhibition of gene expression in postnatal mice. Nat. Genet. 2002; 32: 107-108). Alternatively, the siRNA can be encapsulated into liposomes to facilitate delivery into a cell (Sorensen, D R, et al. Gene silencing by systemic delivery of synthetic siRNAs in adult mice. J Mol Biol. 2003; 327: 761-766). The siRNAs can also be introduced into cells via transient transfection.

RNAi expression vector”, also referred to herein as a “dsRNA-encoding plasmid”, or shRNA-expressing vector, refers to replicable nucleic acid constructs used to express (transcribe) RNA which produces siRNA moieties in the cell in which the construct is expressed. Such vectors include a transcriptional unit comprising an assembly of (1) genetic element(s) having a regulatory role in gene expression, for example, promoters, operators, or enhancers, operatively linked to (2) a “coding” sequence which is transcribed to produce a double-stranded RNA (two RNA moieties that anneal in the cell to form an siRNA, or a single hairpin RNA which can be processed to an siRNA), and (3) appropriate transcription initiation and termination sequences. The choice of promoter and other regulatory elements generally varies according to the intended host cell. In general, expression vectors of utility in recombinant DNA techniques are often in the form of “plasmids” which refer to circular double stranded DNA loops which, wherein the vector may or may not become integrated in the chromosome. In the present specification, “plasmid” and “vector” are used interchangeably as the plasmid is the most commonly used form of vector. The invention contemplates other forms of expression vectors which serve equivalent functions and which become known in the art subsequently.

A number of expression vectors have also been developed to continually express siRNAs in transiently and stably transfected mammalian cells (Brummelkamp et al., 2002, Science 296: 550-553; Sui et al., 2002, PNAS 99(6): 5515-5520; Paul et al., 2002, Nature Biotechnol. 20: 505-508). Some of these vectors have been engineered to express small hairpin RNAs (shRNAs), which get processed in vivo into siRNA-like molecules capable of carrying out gene-specific silencing. In certain embodiments, an shRNA contains plasmid under the control of a promoter, for example a U6 promoter (Paul, C P, et al. Effective expression of small interfering RNA in human cells. Nat. Biotechnol. 2002; 20: 505-508). Another type of siRNA expression vector encodes the sense and antisense siRNA strands under control of separate pol III promoters (Miyagishi and Taira, 2002, Nature Biotechnol. 20: 497-500). The siRNA strands from this vector, like the shRNAs of the other vectors, have 3′ thymidine termination signals. The shRNA gene can be delivered via a suitable vector system, e.g., adenovirus, adeno-associated virus (AAV), or retrovirus (Xia, H, et al. siRNA-mediated gene silencing in vitro and in vivo. Nat. Biotechnol. 2002; 20: 1006-1010; and Barton, G M, et al. Retroviral delivery of small interfering RNA into primary cells. Proc. Natl. Acad. Sci. USA 2002; 99: 14943-14945). In certain embodiments, the invention contemplates use of RNAi vectors which permit stable transfection, and continuous reduction or inhibition of the function of the target protein.

In certain embodiments, the RNA may comprise one or more strands of polymerized ribonucleotide. It may include modifications to either the phosphate-sugar backbone or the nucleoside. For example, the phophodiester linkages of natural RNA may be modified to include at least one of a nitrogen or sulfur heteroatom. Modifications in RNA structure may be tailored to allow specific genetic inhibition while avoiding a general panic response in some organisms which can be generated by dsRNA. Likewise, bases may be modified to block the activity of adenosine deaminase. RNA may be produced enzymatically or by partial/total organic synthesis; any modified ribonucleotide can be introduced by in vitro enzymatic or organic synthesis.

The double-stranded structure may be formed by a single self-complementary RNA strand or two complementary RNA strands. RNA duplex formation may be initiated either inside or outside the cell. The RNA may be introduced in an amount which allows delivery of at least one copy per cell. Higher doses (e.g., at least 5, 10, 100, 500 or 1000 copies per cell) of double-stranded material may yield more effective inhibition; lower doses may also be useful for specific applications. Inhibition is sequence-specific in that nucleotide sequences corresponding to the duplex region of the RNA are targeted for genetic inhibition. The iRNA molecule may be at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 nucleotides in length.

The RNAi constructs contain a nucleotide sequence that hybridizes under physiologic conditions of the cell to the nucleotide sequence of at least a portion of the mRNA transcript for the gene to be inhibited (i.e., the “target” gene). The double-stranded RNA need only be sufficiently similar to natural RNA that it has the ability to mediate RNAi. Thus, the invention has the advantage of being able to tolerate sequence variations that might be expected due to genetic mutation, strain polymorphism or evolutionary divergence. The number of tolerated nucleotide mismatches between the target sequence and the RNAi construct sequence is no more than 1 in 5 basepairs, or 1 in 10 basepairs, or 1 in 20 basepairs, or 1 in 50 basepairs. Mismatches in the center of the siRNA duplex are most critical and may essentially abolish cleavage of the target RNA. In contrast, nucleotides at the 3′ end of the siRNA strand that is complementary to the target RNA do not significantly contribute to specificity of the target recognition.

Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). In certain embodiments, RNA containing a nucleotide sequences identical to a portion of the target gene are suitable for attenuation and/or inhibition target protein activity. In certain embodiments, RNA sequences with insertions, deletions, and single point mutations relative to the target sequence can be effective for inhibition. Thus, one hundred percent sequence identity between the RNA and the target sequence is not required to attenuate and/or inhibit target activity. Sequence identity between the iRNA and the target of about 99%, 98%, 97%, 96%, 95%, 94%, 93%, 92% or 91% is contemplated of the antagonists and the methods of the present invention. Alternatively, the duplex region of the RNA may be defined functionally as a nucleotide sequence that is capable of hybridizing with a portion of the target gene transcript (e.g., 400 mM NaCl, 40 mM PIPES pH 6.4, 1 mM EDTA, 50° C. or 70° C. hybridization for 12-16 hours; followed by washing).

RNA may be synthesized either in vivo or in vitro. Endogenous RNA polymerase of the cell may mediate transcription in vivo, or cloned RNA polymerase can be used for transcription in vivo or in vitro. For transcription from a transgene in vivo or an expression construct, a regulatory region (e.g., promoter, enhancer, silencer, splice donor and acceptor, polyadenylation) may be used to transcribe the RNA strand (or strands). Inhibition may be targeted by specific transcription in an organ, tissue, or cell type; stimulation of an environmental condition (e.g., infection, stress, temperature, chemical inducers); and/or engineering transcription at a developmental stage or age. The RNA strands may or may not be polyadenylated; the RNA strands may or may not be capable of being translated into a polypeptide by a cell's translational apparatus. RNA may be chemically or enzymatically synthesized by manual or automated reactions. The RNA may be synthesized by a cellular RNA polymerase or a bacteriophage RNA polymerase (e.g., T3, T7, SP6). Any suitable method for use and production of an expression construct that are known in the art is contemplated by the present invention as a method to attenuate and/or reduce the target gene activity.

In certain embodiments, the iRNAs are “small interfering RNAs” or “siRNAs.” These nucleic acids can be from about 6 to about 40, 38, 36, 34, 32, 30, 28, 26, 24, 22, 20, 18, 16, 14, 12, 10, 8 nucleotides in length; from about 8 to about 40, 38, 36, 34, 32, 30, 28, 26, 24, 22, 20, 18, 16, 14, 12, 10 nucleotides in length; from about 10 to about 40, 38, 36, 34, 32, 30, 28, 26, 24, 22, 20, 18, 16, 14, 12 nucleotides in length; from about 12 to about 40, 38, 36, 34, 32, 30, 28, 26, 24, 22, 20, 18, 16, 14, nucleotides in length; from about 14 to about 40, 38, 36, 34, 32, 30, 28, 26, 24, 22, 20, 18, 16, nucleotides in length; from about 16 to about 40, 38, 36, 34, 32, 30, 28, 26, 24, 22, 20, 18 nucleotides in length; from about 18 to about 40, 38, 36, 34, 32, 30, 28, 26, 25, 24, 23, 22, 21, 20, 19 nucleotides in length; from about 19 to about 40, 38, 36, 34, 32, 30, 28, 26, 25, 24, 23, 22, 21, 20, nucleotides in length; from about 20 to about 40, 38, 36, 34, 32, 30, 28, 26, 25, 24, 23, 22, 21 nucleotides in length, from about 21 to about 40, 38, 36, 34, 32, 30, 28, 26, 25, 24, 23, 22, nucleotides in length, from about 22 to about 40, 38, 36, 34, 32, 30, 28, 26, 25, 24, 23, nucleotides in length, from about 23 to about 40, 38, 36, 34, 32, 30, 28, 26, 25, 24, nucleotides in length, from about 24 to about 40, 38, 36, 34, 32, 30, 28, 26, 25, nucleotides in length, e.g., corresponding in length to the fragments generated by nuclease “dicing” of longer double-stranded RNAs. The siRNA are double stranded, and may include short overhangs at each end. The overhangs are 1-6 nucleotides in length at the 3′ end. It is known in the art that the siRNAs can be chemically synthesized, or derived from a longer double-stranded RNA or a hairpin RNA. The siRNAs have significant sequence similarity to a target RNA so that the siRNAs can pair to the target RNA and result in sequence-specific degradation of the target RNA through an RNA interference mechanism. The siRNAs are understood to recruit nuclease complexes and guide the complexes to the target mRNA by pairing to the specific sequences. As a result, the target mRNA is degraded by the nucleases in the protein complex. In a particular embodiment, the 21-23 nucleotides siRNA molecules comprise a 3′ hydroxyl group.

The siRNA molecules of the present invention can be obtained using a number of techniques known to those of skill in the art. For example, the siRNA can be chemically synthesized or recombinantly produced using methods known in the art. For example, short sense and antisense RNA oligomers can be synthesized and annealed to form double-stranded RNA structures with 2-nucleotide overhangs at each end (Caplen, et al. (2001) Proc Natl Acad Sci USA, 98:9742-9747; Elbashir, et al. (2001) EMBO J, 20:6877-88). These double-stranded siRNA structures can then be directly introduced to cells, either by passive uptake or a delivery system of choice, such as described below. In certain embodiments, the siRNA constructs can be generated by processing of longer double-stranded RNAs, for example, in the presence of the enzyme dicer. The siRNA molecules can be purified using a number of techniques known to those of skill in the art. For example, gel electrophoresis can be used to purify siRNAs. Alternatively, non-denaturing methods, such as non-denaturing column chromatography, can be used to purify the siRNA. In addition, chromatography (e.g., size exclusion chromatography), glycerol gradient centrifugation, affinity purification with antibody can be used to purify siRNAs.

In certain embodiments, at least one strand of the siRNA molecules has a 3′ overhang from about 1 to about 6 nucleotides in length, or from about 2 to 4 nucleotides in length, or from about 1 to 3 nucleotides in length. In certain embodiments, one strand having a 3′ overhang and the other strand being blunt-ended or also having an overhang. The length of the overhangs may be the same or different for each strand. In order to further enhance the stability of the siRNA, the 3′ overhangs can be stabilized against degradation. In one embodiment, the RNA is stabilized by including purine nucleotides, such as adenosine or guanosine nucleotides. Alternatively, substitution of pyrimidine nucleotides by modified analogues, e.g., substitution of uridine nucleotide 3′ overhangs by 2′-deoxythymidine is tolerated and does not affect the efficiency of RNAi. The absence of a 2′ hydroxyl significantly enhances the nuclease resistance of the overhang in tissue culture medium and may be beneficial in vivo.

In other embodiments, the RNAi construct is in the form of a long double-stranded RNA. In certain embodiments, the RNAi construct is at least 25, 50, 100, 200, 300 or 400 bases. In certain embodiments, the RNAi construct is 400-800 bases in length. The double-stranded RNAs are digested intracellularly, e.g., to produce siRNA sequences in the cell. However, use of long double-stranded RNAs in vivo is not always practical, presumably because of deleterious effects which may be caused by the sequence-independent dsRNA response. In such embodiments, the use of local delivery systems and/or agents which reduce the deleterious effects are preferred.

In certain embodiments, the RNAi construct is in the form of a hairpin structure (i.e., hairpin RNA). The hairpin RNAs can be synthesized exogenously or can be formed by transcribing from RNA polymerase III promoters in vivo. The hairpin structure can vary in length from about 15 to about 25 nucleotides, from about 15 to about 24 nucleotides, from about 15 to about 23 nucleotides, from about 15 to about 22 nucleotides, from about 15 to about 21 nucleotides, from about 15 to about 20 nucleotides, from about 15 to about 19 nucleotides, from about 15 to about 18 nucleotides, from about 15 to about 17 nucleotides, from about 15 to about 16 nucleotides. Examples of making and using such hairpin RNAs for gene silencing in mammalian cells are described in, for example, Paddison et al., Genes Dev, 2002, 16:948-58; McCaffrey et al., Nature, 2002, 418:38-9; McManus et al., RNA, 2002, 8:842-50; Yu et al., Proc Natl Acad Sci USA, 2002, 99:6047-52). In certain embodiments, such hairpin RNAs are engineered in cells or in an animal to ensure continuous and stable suppression of a desired gene. It is known in the art that siRNAs can be produced by processing a hairpin RNA in the cell.

In yet other embodiments, a plasmid is used to deliver the double-stranded RNA, e.g., as a transcriptional product. In such embodiments, the plasmid is designed to include a “coding sequence” for each of the sense and antisense strands of the RNAi construct. The coding sequences can be the same sequence, e.g., flanked by inverted promoters, or can be two separate sequences each under transcriptional control of separate promoters. After the coding sequence is transcribed, the complementary RNA transcripts base-pair to form the double-stranded RNA.

According to the invention antagonist or inhibitors of LIS1, dynein, dynactin and LIS1-, dynein-, dynactin-mediated pathway can also include “dominant negative” molecules and constructs, including polypeptides derived from the amino acid sequence of LIS1, dynein, or dynactin. In one aspect, a dominant negative molecule interferes with, and/or inhibits the function or activity of the wild-type protein in a cell. In one aspect, a dominant negative polypeptide can be an inactive variant of a polypeptide, which, by interacting with the cellular machinery, and/or other components of a (multi)protein complex, displaces an active polypeptide from its interaction with the cellular machinery, and/or a multiprotein complex, and/or competes with the active polypeptide, thereby reducing the effect of the active polypeptide. For example, a dominant negative polypeptide which is a truncated version of the active polypeptide may retain interaction with certain binding partners, while the truncated portion may lead to loss of interaction with other binding partners, and/or loss of a particular protein function. Overexpression of a full length polypeptide which is a member of (multi)protein complex may also act as a dominant negative polypeptide by virtue of polypeptide abundance in non-stoichiometric amounts relative to its binding partners, and/or other components of a multiprotein complex. The more abundant polypeptide can interact with its binding partner(s) outside of a multiprotein complex, thus titrating, preventing and/or displacing such binding partners from inclusion in a functional multiprotein complex.

In other aspects, the invention provides dominant negative constructs directed to any one of LIS1, dynein, dynactin, or any one of their positive regulators, and/or activity mediators. The concept of dominant negative constructs which inhibit activity of a wild-type target, and/or the pathway in which the target functions is well known in the art. In certain aspects, dominant negative constructs can be generated by expression of fusion polypeptides and/or proteins which comprise a tag, label, for example a fluorescent protein, or any other protein fragment fused to the target protein. In other aspects, dominant negative constructs can be generated by expression or overexpression of protein fragments, for example but not limited to specific domains that may interact with binding partners, derived from the target protein. In other aspects, dominant negative constructs against a target can be generated by expression of protein fragments and/or variants which have altered binding and/or interaction with proteins in the same functional pathway, which may directly interact with the target. Mutagenesis and screening methods to identify such fragments and/or variants are well known in the art. In other aspects, dominant negative forms can be any other construct and/or fragment which can interfere with the function of the target protein.

In certain aspects, agents which can be used as dominant negative constructs and molecules include but are not limited to: LIS1 full length (LIS1-FL), LIS1 encoding amino acids positions 1-87 of the NH₂-terminal end (LIS1-N), LIS1 encoding amino acids positions 88-410 (LIS1-C), Dynamitin full length. In certain embodiments, human LIS1 full length can be used as a dominant negative molecule. In other embodiments, the N-terminal end from amino acid 1 to amino acid 87 of human LIS1 can be used as a dominant negative molecule. In other embodiments, the C-terminal end, starting at amino acid position 88 to amino acid position 410 of human LIS1 can be used as a dominant negative construct. In other embodiments, human dynamitin full length can be used as a dominant negative molecule. Exemplary sequences which encode dominant negative constructs of the invention are given by SEQ ID NOS: 13, 14, 15, 16. One of ordinary skill in the art appreciates that due to the redundancy of the genetic code, amino acid sequences which function as dominant negative constructs of the invention can be encoded by several different nucleic acid sequences.

Methods for treating tumors, specifically gliomas: In one aspect, the invention provides a method for inhibiting cell division, cell proliferation and/or migration of neural progenitor cells, wherein the method comprises contacting a neural progenitor cell with an antagonist that interferes with the function of LIS1, dynein, or dynactin. In other aspects, the invention provides methods for modulating cell division, proliferation, migration and/or survival of cells, for example but not limited to neural progenitor cells, the method comprising contacting a neural progenitor cells with an antagonist that interferes with the function of LIS1, dynein, or dynactin. In other aspects, the invention provides methods for modulating proliferation, migration and/or survival of tumor cells of neural lineage, the method comprising contacting a cell of neural lineage with an antagonist that interferes with the function of LIS1, dynein, or dynactin.

In another aspect, the invention provides a method for inhibiting morphogenesis of neural progenitor cells, wherein the method comprises contacting a molecule that interferes with the function of LIS1 or dynein to a neural progenitor cell.

In another aspect, the invention provides a method for inhibiting migration of neural progenitor cells, wherein the method comprises contacting a molecule or an agent that interferes with the function of LIS1, dynein, or dynactin, or a positive regulator thereof, to a neural progenitor cell. In certain embodiments, the agent is selected from the group consisting of iRNA molecules, dominant negative constructs, vectors and/or plasmids which express iRNAs or dominant negative constructs, compounds which inhibit the activity of LIS1 or dynein, or a positive regulator thereof.

In another aspect, the invention provides a method for treating disorders associated with abnormal growth or migration of neural progenitor cells, wherein the method comprises administering to a subject an effective amount of a composition that comprises molecules that interfere with the function of dynein, dynactin or LIS1. In other aspects, the invention provides methods for treating disorders associated with abnormal growth of cells of neural lineage, wherein the method comprises administering to a subject an effective amount of a composition that comprises molecules that interfere with the function of dynein, dynactin or LIS1. In certain aspects, the disorder associated with abnormal growth is a glioma. In certain aspects, the disorder associated with abnormal growth is a brain tumor.

The methods of treatment and prevention of the present invention can be used in combination with any other anti-tumor therapy, including specific anti-glioma therapies. In non-limiting examples of a glioma treatment, the treatment may comprise surgery, administration of any additional anti-glioma compound and/or radiation therapy.

Pharmaceutical compositions: In another aspect, the invention provides compositions comprising any one of the antagonists of the present invention. In another aspect, the invention provides compositions comprising therapeutically effective amounts of any one of the antagonists of the present invention. The term “therapeutically effective amount” used interchangeably with the term “effective amount” as used herein means that amount of a compound, material, such as the agonists or antagonists of the present invention, or composition comprising a compound of the present invention which is effective for producing some desired therapeutic effect by modulating, increasing or decreasing, LIS1, dynactin, dynein mediated activity in at least a sub-population of cells in a subject, and thereby modulating the biological consequences of that pathway in the treated cells, at a reasonable benefit/risk ratio applicable to any medical treatment.

The pharmaceutical compositions comprise any suitable pharmaceutically acceptable material, which can be an excipient, carrier or delivery agent. As used herein the term “pharmaceutically acceptable material” means a composition or vehicle, such as a liquid or solid filler, diluent, excipient, solvent or encapsulating material, involved in carrying or transporting the subject antagonists from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

In certain aspects, the pharmaceutical compositions comprise antagonist, including but not limited to iRNA antagonists, of LIS1, dynein, dynactin, or any one of their positive regulators. In certain aspects, the compositions of the invention can comprise naked siRNA, and/or a vehicle, for example a recombinant plasmid or vector which expresses the siRNA, a liposome, polymer or a nanoparticle comprising siRNA. In other aspects, the compositions of the invention can comprise a vehicle, for example a plasmid or a vector which encode antagonist, such as a dominant negative form of the target, or a liposome, polymer or a nanoparticle comprising a dominant negative form.

The pharmaceutical compositions of the present invention can be formulated in solid dosage form, liquid dosage forms, suspensions, or aerosols. Methods for preparing various formulations and dosage forms suitable for delivery of the compositions of the invention are known in the art and are contemplated by the invention. In another aspect, the pharmaceutical compositions of the present invention can be coated on medical devices which can be used for treatment of brain tumors. In certain aspects, the subject invention provides a medical device comprising a coating adhered to at least one surface, wherein the coating comprises polymer matrix and an RNAi construct or dominant negative constructs as disclosed herein. Optionally the coating further comprises protein noncovalently associated with the RNAi construct (or selected to interact with the RNAi construct upon release from the coating). Such coatings can be applied to surgical implements or devices which can be used for treatment of brain tumors. In certain aspects, the invention provides medical devices such as wafers which can be coated with a composition comprising antagonist or against of the invention.

The compositions comprising antagonist of the present invention are administered in a therapeutically effective amount, which can be readily determined by one of skill in the art. In certain aspects, therapeutic amount is an amount which leads to inhibition of the target protein level and/or activity. The amount of antagonist which can produce inhibition of the target protein can vary depending on the level of inhibition which is desired. Routine optimization may be involved to ensure that the antagonist can produce the desired level of inhibition. In certain aspects, the level of inhibition can vary from about 95% to about 10% inhibition of the level and/or activity of the target nucleic acid, or protein. In certain aspects, the level of inhibition can vary: from about 95% to about 10%, 15%, 20%, 25%, 30%, 35%, 40%, 45%, 50%, 55%, 60%, 65%, 70%, 75%, 80%, 85%, 90% inhibition of the level and/or activity of the target protein; from about 90% to about 10%, 15%, 20%, 25%, 30%, 35%, 40%, 45%, 50%, 55%, 60%, 65%, 70%, 75%, 80%, 85% inhibition of the level and/or activity of the target protein; from about 85% to about 10%, 15%, 20%, 25%, 30%, 35%, 40%, 45%, 50%, 55%, 60%, 65%, 70%, 75%, 80% inhibition of the level and/or activity of the target protein; from about 80% to about 10%, 15%, 20%, 25%, 30%, 35%, 40%, 45%, 50%, 55%, 60%, 65%, 70%, 75% inhibition of the level and/or activity of the target protein; from about 75% to about 10%, 15%, 20%, 25%, 30%, 35%, 40%, 45%, 50%, 55%, 60%, 65%, 70%, 75%, 70% inhibition of the level and/or activity of the target protein; from about 70% to about 10%, 15%, 20%, 25%, 30%, 35%, 40%, 45%, 50%, 55%, 60%, 65%, inhibition of the level and/or activity of the target protein; from about 65% to about 10%, 15%, 20%, 25%, 30%, 35%, 40%, 45%, 50%, 55%, 60% inhibition of the level and/or activity of the target protein; from about 60% to about 10%, 15%, 20%, 25%, 30%, 35%, 40%, 45%, 50%, 55%, from about 55% to about 10%, 15%, 20%, 25%, 30%, 35%, 40%, 45%, 50%, from about 50% to about 10%, 15%, 20%, 25%, 30%, 35%, 40%, 45%, from about 45% to about 10%, 15%, 20%, 25%, 30%, 35%, 40%, from about 40% to about 10%, 15%, 20%, 25%, 30%, 35%, from about 35% to about 10%, 15%, 20%, 25%, 30%, from about 30% to about 10%, 15%, 20%, 25%, from about 25% to about 10%, 15%, 20%, from about 20% to about 10%, 15%, 15% to about 10%, inhibition of the level and/or activity of the target protein. In certain aspects, the level of inhibition can be about 99%, 98%, 95%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20% inhibition of the level and/or activity of the target protein. In certain aspects, antagonist directed to LIS1, dynein or dynactin, can be co-administered.

Methods of administration: The compositions of the present invention can be administered to a subject by any suitable method. The pharmaceutical compositions of the present invention may be administered in a number of ways depending upon whether local or systemic treatment is desired and upon the area to be treated. Administration may be topical (including ophthalmic and to mucous membranes including vaginal and rectal delivery, pulmonary, e.g. by inhalation of powders or aerosols, including by nebulizer; intratracheal, intranasal, epidermal and transdermal, oral or parenteral. Parenteral administration includes intravenous, intra-arterial, subcutaneous, intraorbital, intracapsular, intraspinal, intrasternal, intraperitoneal or intramuscular injection or infusion; or intracranial, e.g., intrathecal or intraventricular etc., administration.

In certain aspect, the compositions are administered systemically via injection and/or infusion. In certain aspect, the compositions are administered via injection and/or infusion to the tumor site. In certain aspects, systemic administration of the compositions includes but is not limited to intracranial delivery. In other aspects, the compositions are delivered by intracerebroventricular administration.

The present invention contemplates any genetic manipulation for use in modulating expression and/or activity of LIS1, dynein, dynactin or any other member of the dynein pathway. Examples of genetic manipulation include, but are not limited to, expression of antisense constructs with or without inducible promoters, and the like. Delivery of nucleic acid construct to cell in vitro or in vivo may be conducted using any suitable method which introduces the nucleic acid construct into the cell such that the desired event occurs (e.g. reduction of target gene expression and/or inhibition of target protein activity). Delivery of antagonist carrying genetic information into cells is achieved by any of various methods including, but not limited to, directed injection of naked DNA constructs, bombardment with gold particles loaded with expression constructs, and macromolecule mediated gene transfer using, for example, liposomes, biopolymers, dendrimers, and the like, as delivery vehicles for the antagonists of the present invention. In certain aspect, delivery vehicles can be targeted to glioma cells by recognition of specific cell markers, this delivering an agent that interferes with LIS-1, Dynein, Dynactin function specifically to glioma cells.

In certain aspects, methods for administration use gene delivery vehicles derived from viruses, including, but not limited to, adenoviruses, retroviruses, poxviruses, vaccinia viruses, and adeno-associated viruses. Because of the higher efficiency as compared to retroviruses, vectors derived from adenoviruses can be more suitable as gene delivery vehicles for transferring nucleic acid molecules into host cells in vivo. Adenoviral vectors have been shown to provide very efficient in vivo gene transfer into a variety of solid tumors in animal models and into human solid tumor xenografts in immune-deficient mice. Examples of viral vectors, including adenoviral vectors and methods for gene transfer are well known in the art and are herein incorporated. In certain aspects, replication-deficient polio vector can be used to deliver the inhibitors of the invention. Such polio vectors can infect neural cells, integrate into the genome, and deliver the iRNA without being infectious.

Vectors may be administered to subjects in a variety of ways. In certain aspects of the present invention, vectors are administered into tumors or tissue associated with tumors using direct injection. In other embodiments, administration is via the blood or lymphatic circulation. Vector doses which can be administered to a subject during treatment can be readily determined by a skilled artisan. For example vector doses can be determined based on in vitro or animal studies.

Screening methods: In yet another aspect, the invention provides a method for identifying an agent that interferes with the function of LIS1, dynactin, or dynein, or LIS1-, dynactin-, or dynein-mediated activity. In one embodiment, an agent can be a nucleic acid. In another embodiment, an agent can be a small molecule. The invention provides methods where there is an interference with LIS1 dynactin, dynein mediated activity which results in arrest of neuronal migration at the multipolar stage and interferes with neocortial expansion.

Another aspect of the invention is a method for identifying agents that modulate, increase or decrease, a LIS1, dynein, or dynactin mediated activity in a cell (such as but not limited to a neural progenitor cell, a glial cell, or a glioma cell), the method comprising: a) contacting a cell that expresses LIS1, dynein, or dynactin (e.g., a glioma cell) with an agent, under conditions effective for the agent to exert an effect on LIS1, dynein, or dynactin expression and/or activity in the cell; and b) determining the level of LIS1, dynein, or dynactin expression and/or activity in the contacted cell compared to a baseline value determined from a cell which has not been contacted with the agent, wherein an agent that elicits a decrease in the amount and/or activity of LIS1, dynein, or dynactin in the contacted cell compared to the baseline value is a candidate for an antagonist of LIS1, which can also be an anti-glioma agent. In other aspects, an agent that elicits an increase in the amount and/or activity of LIS1, dynein, or dynactin in the contacted cell compared to the baseline value is a candidate for an agonist of LIS1, dynein, or dynactin. In certain aspect, determining step can be done by any suitable method known in the art, including but not limited to any one of the methods described herein, such as measuring protein levels, functional determination such as cell cycle progression, cell proliferation, and/or migration of cells. In certain aspects, the method optionally further comprises: c) determining if the candidate elicits an anti-glioma effect in vivo (e.g. determining if it inhibits growth, migration, invasion and/or metastasis of a glioma tumor in an animal).

Suitable transfected cells for the above method (or for other methods of the invention) include any eukaryotic cell (e.g. a CHO cell or an HEK-293 cell) which expresses a full length, fragment, variant, mimetic or analog of LIS1, dynein, or dynactin. The term “a fragment, variant, mimetic or analog” of LIS1, dynein, or dynactin includes any one of a number of LIS1, dynein, or dynactin variants molecules, provided that the modified protein retains at least one of the activities of the wild type protein. Modified proteins can take the form of, e.g. conservative amino acid substitutions, deletions, additions, protein fusions, etc. and include naturally occurring allelic variants. Suitable types of modified proteins will be evident to one of ordinary skill in the art. Neural progenitor cell, glial cells, and/or glioma cells that express LIS1, dynein, or dynactin can also be used in the method. The above method to identify an agent which increases, decreases, or inhibits a LIS1, dynein, or dynactin—mediated activity of a cell can be performed in vitro, or in vivo. In certain aspects, the activity that is assayed is cell cycle progression, which is easily monitored. For example, one can readily assay antagonist agents in a high throughput assays. Subsequently, promising candidates can then optionally be screened for secondary activities, such as migratory behavior, or any other suitable phenotype.

Binding of agents to LIS1, dynein, or dynactin can also be assayed. Promising candidates can subsequently be tested in vivo, e.g in an animal model for glioma. Finally, the agent can be tested in a non-human primate or a human. The order and numbering of the steps in the methods described herein are not meant to imply that the steps of any method described herein must be performed in the order in which the steps are listed or in the order in which the steps are numbered.

The steps of any method disclosed herein can be performed in any order which results in a functional method. In some embodiments, the method is performed with some or all of the steps carried out simultaneously. The screening methods of the invention can be adapted to any one of a variety of high throughput methods, wherein some of the steps can be performed automatically. In the assays described herein, an antagonist agent may or may not inhibit expression of LIS1, dynein, or dynactin mediated activity. In certain aspects, the invention relates to methods to determine if an agent functionally modulates a target protein activity to produce a certain phenotype which is associated with inhibition of LIS1, dynein, or dynactin mediated activity, irrespective of whether an inhibition of target expression is detected. A variety of classes of antagonist can be tested by this screening method, including antisense nucleic acids, siRNAs, inhibitors, antibodies, etc. In certain aspects, the method is used to test small molecule compounds, e.g. compounds from a combinatorial library. In certain aspects, the method is used to test libraries of siRNAs.

In certain aspects the method for screening can be used to test iRNAs directed to LIS1, dynein, or dynactin mediated activity. Methods to introduce polynucleotides, or peptides, of the invention into cells (to “contact” the cells), are well known in the art. Such methods include, e.g. transfection (e.g., mediated by DEAE-Dextran or calcium phosphate precipitation), infection via a viral vector (e.g., retrovirus, adenovirus, adeno-associated virus, lentivirus, pseudotyped retrovirus, poxvirus or vaccinia vectors), injection, such as microinjection, electroporation, sonoporation, a gene gun, liposome delivery (e.g., Lipofectin, Lipofectamine (GIBCO-BRL, Inc., Gaithersburg, Md.), Superfect (Qiagen, Inc. Hilden, Germany) and Transfectam (Promega Biotec, Inc., Madison, Wis.), or other liposomes developed according to procedures standard in the art), or receptor-mediated uptake and other endocytosis mechanisms.

### EXAMPLES

RNAi and dominant negative constructs: For RNAi, shRNA constructs based on the pRNAT-U6.1/Neo vector (GenScript), which expresses a GFP marker along with a short hairpin siRNA, and Cy3-labeled synthetic siRNA oligonucleotides (Dharmacon) were used. Targeting sequences in the shRNA plasmid pRNAT-LIS1 is the following: 5′-GAGAUGAACUAAAUCGAGCUA-3′ referred to as SEQ ID NO: 1, and the siRNA oligonucleotide duplex Cy3-5′-GAACAAGCGAUGCAUGAAGdTdT-3′ referred to as SEQ ID NO: 2, were chosen from a portion of the LIS1-coding region, where the sequence is identical among humans, African green monkeys, cows, rats, and mice. A triple mutated sequence in the pRNAT-LIS1 construct (pRNAT-LIS1-3mt; 5′-GACAUCAAGUAAAUCGAGCUA-3′ referred to as SEQ ID NO: 3) and a scrambled sequence of the same GC contents (Cy3-5′-AUUGUAUGCGAUCGCAGACdTdT-3′ referred to as SEQ ID NO: 4) are used as controls. No other related sequences were found in other sequences in the genome of these species.

Another example of iRNA sequence targeting LIS1 is the following: 5′-GGATGCTACAATTAAGGTGTG-3′ (referred to as SEQ ID NO: 5). An example of shRNA sequence targeting dynein heavy chain is the following: 5′-AGGCTTTAACCAAGCAGATAA-3′ (referred to as SEQ ID NO: 6). The siRNAs were chosen to be identical, at least, between human, monkey, cow, rat and mouse. The siRNAs of the invention were tested in monkey and rat cell lines. It is expected that these siRNAs will be effective in human, cow, and mouse cells. Thus, the iRNAs of the invention are expected to inhibit human target genes.

To construct dominant negative LIS1-GFP fusions, full-length African green monkey LIS1 (Faulkner et al., 2000) and a sequence encoding the first 87 NH₂-terminal aa (Tai et al., 2002) were made by PCR (primers: forward referred to as SEQ ID NO: 7, 5′-GGAAGATCTCCAGGAATTCTGCTGTCCC-3′; LIS1 reverse referred to as SEQ ID NO: 8, 5′-TAACCGCGGCCGCTCAACG-3′; and LIS1N reverse referred to as SEQ ID NO: 9, 5′-TAACCGCGGCCGCTACTGACC-3′) using PfuTurbo DNA polymerase (Stratagene). The PCR products were then restriction enzyme digested and shuttled into BglII and SacII sites of pEGFP-C1 (CLONTECH Laboratories, Inc.).

In vitro RNAi assay: For transient transfection with pRNAT constructs, cells were plated on six-well dishes to 70-80% confluency and were transfected using LipofectAMINE 2000 (Invitrogen). For transient transfection with siRNA oligonucleotides, cells were plated on six-well dishes to 40-50% confluency and were transfected using OligofectAMINE (Invitrogen). Cells were lysed 45-48 h after transfection in 50 mM Tris-HCl, pH 8.0, 150 mM NaCl, 1% NP-40, and 1 mM EGTA with protease inhibitor cocktail for mammalian tissues (Sigma-Aldrich). pAb anti-LIS1 (Santa Cruz Biotechnology, Inc.) and mAb antitubulin (Sigma-Aldrich) were used for Western blotting.

In utero electroporation: Plasmids or oligonucleotides were transfected using intraventricular injection followed by in utero electroporation (Saito and Nakatsuji, 2001; Tabata and Nakajima, 2001). In brief, pregnant Sprague Dawley rats (Taconic) were used, and 1-2 μl cDNA (1-5 μg/μl) or 1 μg/μl siRNA were injected into the ventricle of embryonic brains at E16. A pair of copper alloy oval plates that were attached to the electroporation generator (Harvard Apparatus) transmitted five electric pulses at 50 V for 50 ms at 1-s intervals through the uterine wall. Animals were maintained according to protocols approved by the Institutional Animal Care and Use Committee at Columbia University.

Immunocytochemistry: Rat embryos were perfused transcardially with ice-chilled saline followed by 4% PFA (EMS) in 0.1 M PBS, pH7.4. Brains were postfixed in PFA overnight and sectioned on a Vibrotome (Ted Pella). Slices were blocked at RT for 1 h with 10% serum, 0.1% Triton X-100, and 0.2% gelatin in PBS. Primary antibodies were applied overnight at the following concentrations: anti-LIS1, 1:200 (Mizuguchi et al., 1995); anti-TuJ1, 1:40 (Babco); antinestin, 1:200 (Chemicon), antivimentin, 1:40 (CBL); 4A4, 1:1,000 (MBL International Corporation); and anti-Ki67, 1:200 (Chemicon). Sections were then washed with PBS and incubated in Cy5-conjugated secondary antibodies (1:200; Jackson ImmunoResearch Laboratories).

Confocal microscopy: Sections were imaged on an inverted laser-scanning confocal microscope (FluoView 300; Olympus) with a 40×NA 0.8 water immersion objective (Olympus). Excitation/emission wavelengths were 488/515 nm (GFP), 568/590 nm (DsRed), and 633/690 (Cy5). Z-series images were collected at 2-3-μm steps in FluoView, and a projection of each stack was used for producing figures. Images were contrast enhanced, assembled into montages, and false color was applied using Photoshop (Adobe). In each brain slice, 100-500 cells could be found positive to GFP when electroporated with the constructs used. When Cy3-labeled synthetic siRNA oligonucleotides were used, 30-80 siRNA-positive cells per slice could be detected. These cells were all counted for statistic analysis (Table 1). In experiments that involved colabeling or cell/process counting, images from individual optical sections were carefully examined.

Live cell imaging: Coronal slices were prepared 24-48 h after electroporation. Slices were placed on Millicell-CM inserts (Millipore) in culture medium containing 25% Hanks balanced salt solution, 47% basal MEM, 25% normal horse serum, 1× penicillin/streptomycin/glutamine (GIBCO BRL), and 0.66% glucose and were incubated at 37° C. in 5% CO₂. Multiple GFP-positive cells (Table 2) were imaged on an inverted microscope (model DMIRB; Leica) with a 40×NA 0.55 objective. Time-lapse images were captured by camera (CoolSNAP HQ; Roper Scientific) using MetaMorph software (Universal Imaging Corp.) at intervals of 10 or 15 min for 10-18 h. Epifluorescence images from several focal planes were deconvolved using AutoDeblur software (AutoQuant Imaging, Inc.) to produce sharp images.

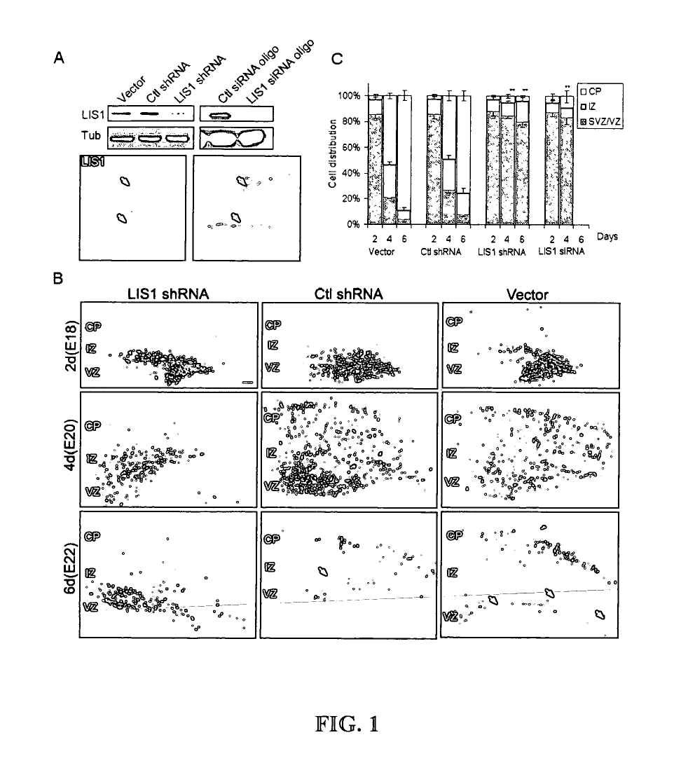

Altered distribution of neuronal cells resulting from LIS1 inhibition: Studies of mouse strains that are heterozygous for LIS1 have revealed a human lissencephaly-like brain disorganization phenotype (Hirotsune et al., 1998; Cahana et al., 2001; Gambello et al., 2003). RNAi and dominant negative cDNAs were used to explore a more complete range of LIS1 defects, and to do so in a wild-type background, and to reveal cell-autonomous effects. For RNAi, both synthetic Cy3-labeled oligonucleotides and cDNAs based on the pRNAT-U6.1/Neo vector, which encodes a short hairpin RNA (shRNA) and coral GFP, were used. The shRNA and small interference RNA (siRNA) sequences correspond to two different regions within LIS1, which are each conserved among multiple vertebrate species. To test the effectiveness of the shRNA construct and siRNA oligonucleotide on LIS1 expression, COS7 cells were transfected, lysed after 48 h and immunoblotted using anti-LIS1 antibody (FIG. 1A, top). Most of the LIS1 protein was absent compared with control cells that were transfected with triple point mutant shRNA, empty vector, or scrambled siRNA oligonucleotide, respectively. Brain sections were also stained with anti-LIS1 antibody 2 days after in utero electroporation with LIS1 shRNA vector and found the LIS1 signal to be absent in transfected cells (FIG. 1A, bottom).

To examine the effects of LIS1 RNAi on brain development, LIS1 shRNA, control shRNA constructs, or empty vector were introduced into neural progenitor cells in rat neocortex by in utero electroporation at embryonic day (E) 16. GFP-positive cells in the VZ/SVZ, intermediate zone (IZ), or cortical plate (CP) were examined 2,4, and 6 d later (FIG. 1, B and C; and Table 1). At day 2, cells that were transfected with LIS1 shRNA, control shRNA, and empty vector were largely found in the VZ and SVZ (88.2±3.1%, n=4 embryos; 86.5±3.7%, n=3 embryos; and 85.2±2.9%, n=4 embryos; respectively) with no obvious differences in spatial distribution between LIS1 shRNA- and control shRNA-transfected brains. By day 4, about half of GFP-positive control cells (control shRNA: 48.7±3.9%, n=3 embryos; empty vector: 53.1±2.2%, n=5 embryos) had become redistributed to the CP. By day 6, the majority of control cells had migrated to the CP (control shRNA: 75.3±4.0%, n=3 embryos; empty vector: 89.0±3.9%, n=3 embryos). The time course of redistribution in control neurons is consistent with previous observations in cells that were labeled with GFP that by using retroviral infection (Noctor et al., 2004) or in utero electroporation (Table 1).

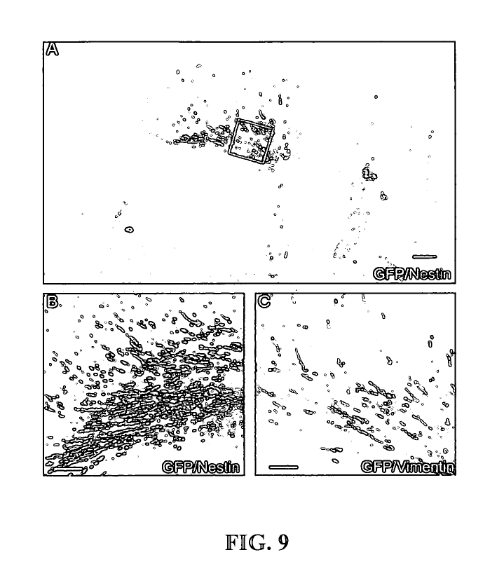

In brains that were transfected with the LIS1 shRNA construct, however, the majority of transfected cells were still distributed in the VZ/SVZ at day 4 (84.9±2.0%, n 5 embryos), with only a subset having reached the lower region of the IZ (10.4±0.8%, n=5 embryos). By day 6, cells expressing LIS1 shRNA had an almost identical distribution to that observed at days 2 and 4 (80.1±4.0% in VZ/SVZ; 16.2±3.3% in IZ; n=3 embryos). Thus, the knockdown of LIS1 by RNAi in neurons had a direct effect on radial redistribution to the CP. A virtually identical migration arrest was produced by LIS1 siRNA oligonucleotide at days 2 and 4 (FIG. 1C). However, the signal of the fluorescent siRNA oligonucleotide was too weak to detect by day 6, probably as a result of degradation or dilution of the fluorochrome or RNA. It can be noted that the redistribution of LIS1 shRNA-expressing cells was inhibited not only at the center of the neocortex but also at its lateral boundaries. In these regions, the radial glial fibers are themselves distorted (FIG. 9; Misson et al., 1991), and migration along these tracks likely contributes to cortical expansion.

Full-length LIS1 (GFP-LIS1), an NH₂-terminal LIS1 fragment (GFP-LIS1N), and the full-length dynamitin subunit of the dynactin complex (GFP-p50), each of which produce dominant inhibitory effects on dynein function in cultured cells were also introduced in cells (Echeverri et al., 1996; Faulkner et al., 2000; Tai et al., 2002). No clear effect on overall cell distribution was observed 2 d after the expression of each of these constructs, but a marked decrease in transfected cell number was observed by day 4. These results indicate that prolonged overexpression of these cDNA constructs may be lethal.

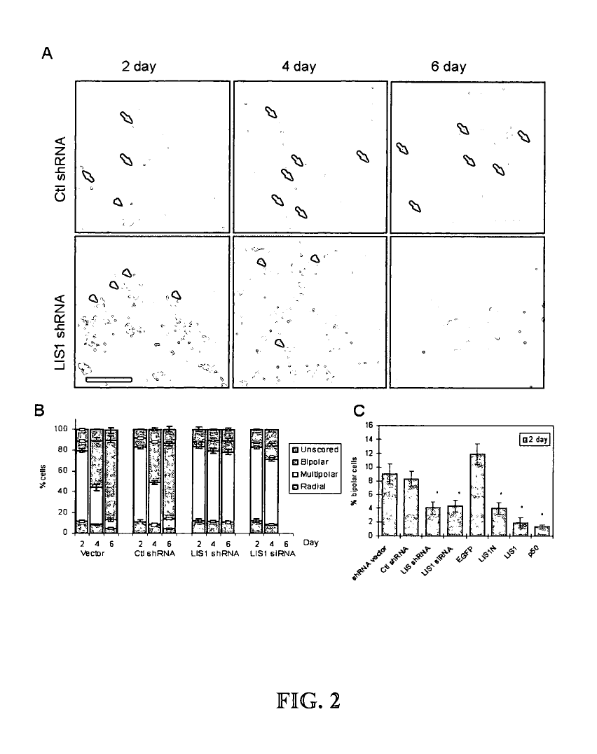

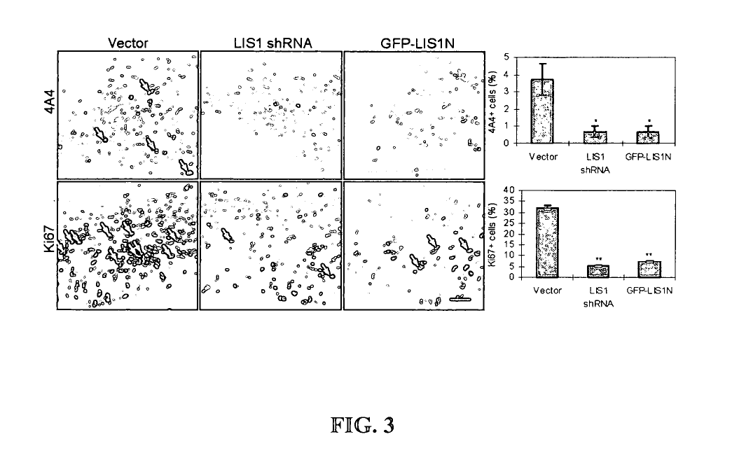

Accumulation of LIS1 and dynactin inhibited cells at the multipolar stage: As noted previously, newborn neurons have been shown to move from the VZ to the SVZ and assume an immobile multipolar morphology before converting to a bipolar morphology and recommencing their radial migration (Tabata and Nakajima, 2003; Noctor et al., 2004). To investigate the involvement of LIS1 in the specific stages in this pathway, morphological analysis of the transfected cells was performed. Individual radial glial, multipolar, and bipolar cells in different regions were readily distinguishable from the analysis of serial confocal images of brain slices (FIG. 2 A). In control brains, many multipolar cells were produced by day 2, constituting a majority of the population in the SVZ (control shRNA: 72.2±1.7%, n=4 embryos; empty vector: 68.3÷1.5%, n=3 embryos; FIG. 2B). Some cells in the lower SVZ and VZ still showed a radial glial morphology (control shRNA: 10.9±2.1%, n=4 embryos; empty vector: 11.4±1.0%, n=3 embryos). Some cells in the upper SVZ and a few cells that had reached the IZ exhibited prominent leading processes extending toward the CP (FIG. 2 A) as expected for bipolar migratory neurons (control shRNA: 9.0±1.4%, n=5; empty vector: 8.3±1.1%, n=3; FIG. 2B). By days 4 and 6, the numbers of cells with radial glial and multipolar morphology were decreased, the majority of control cells had become bipolar (control shRNA: 38.6±1.4%, n=3 embryos and 72.6±1.8%, n=3 embryos; empty vector: 44.6±3.3%, n=5 embryos and 76.8±3.9%, n=3 embryos, respectively; FIG. 2 B), and many had migrated to the CP.

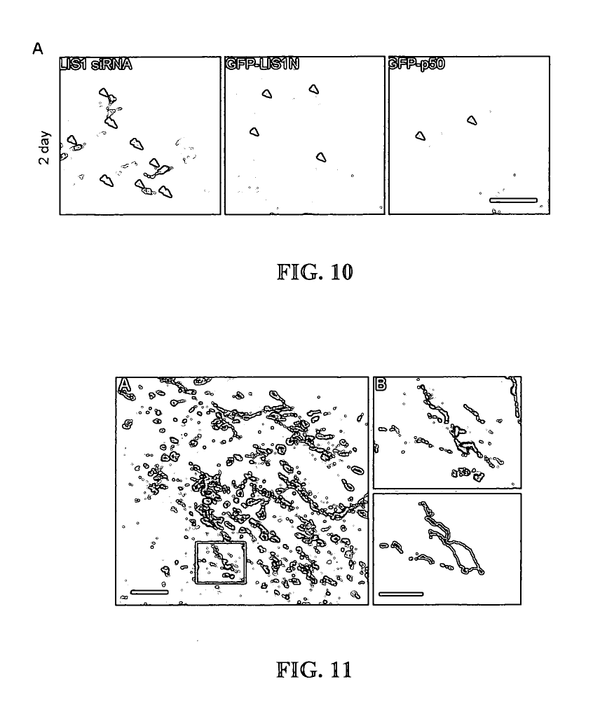

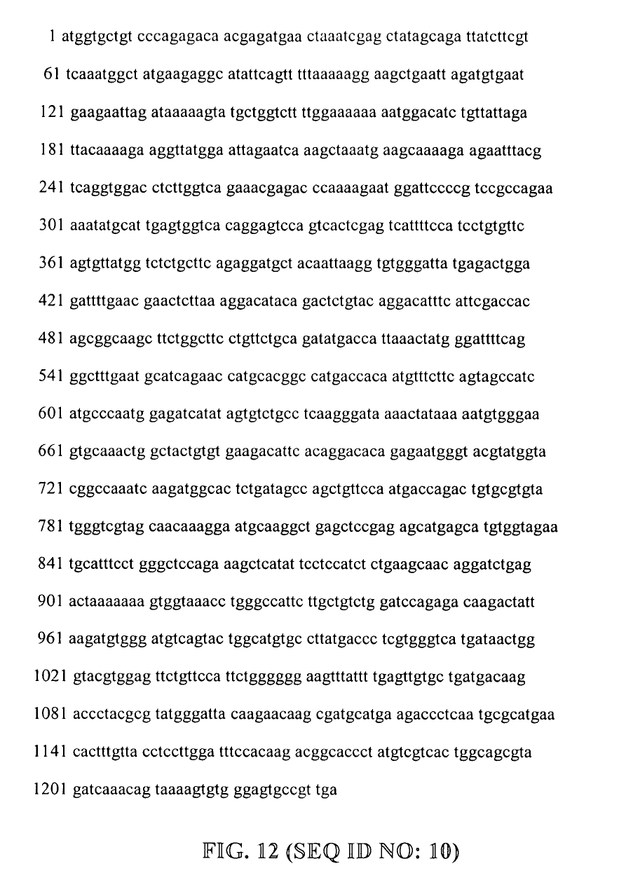

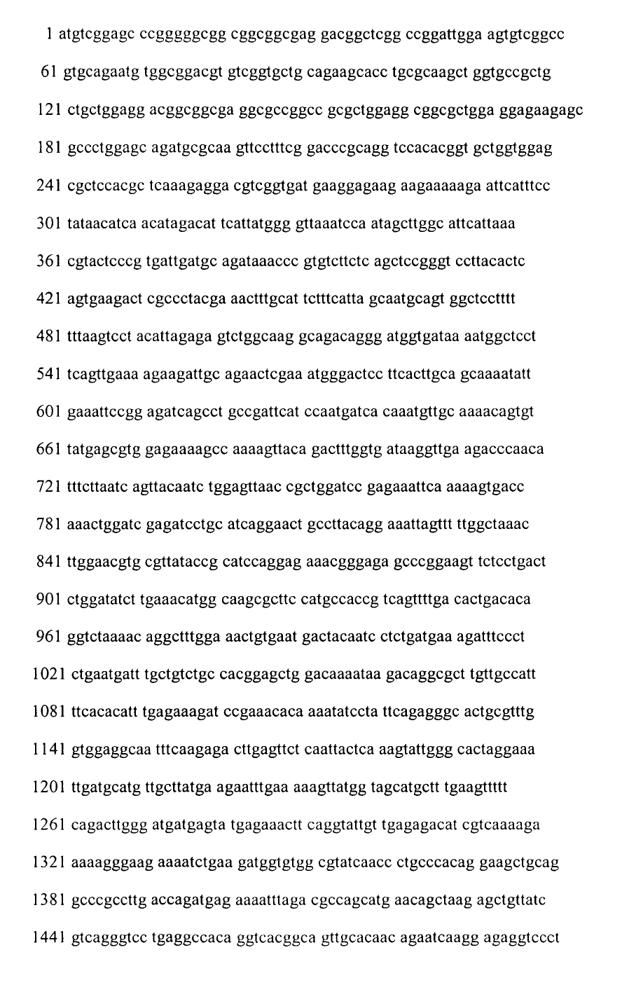

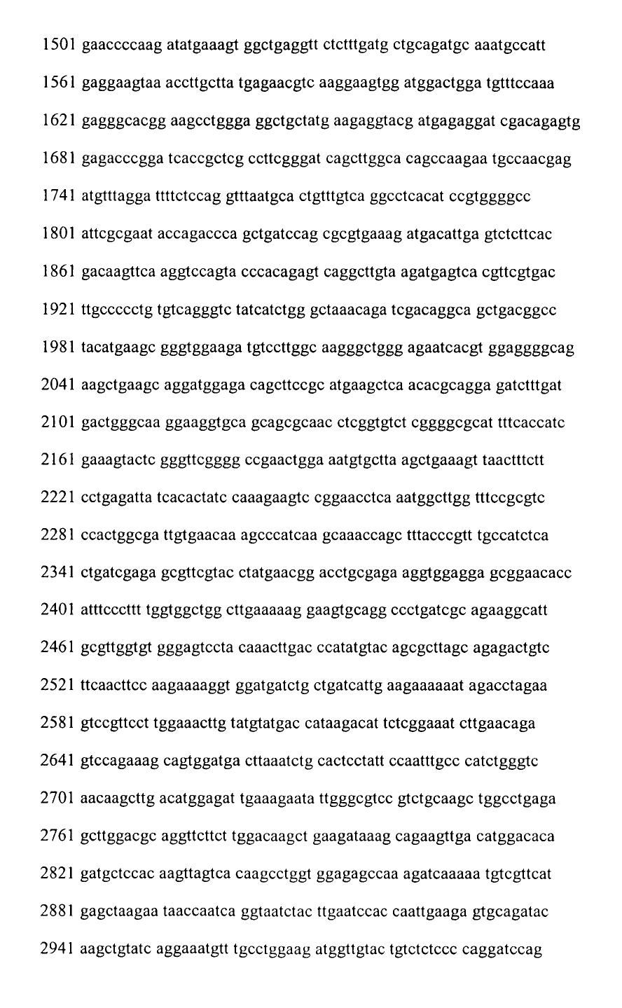

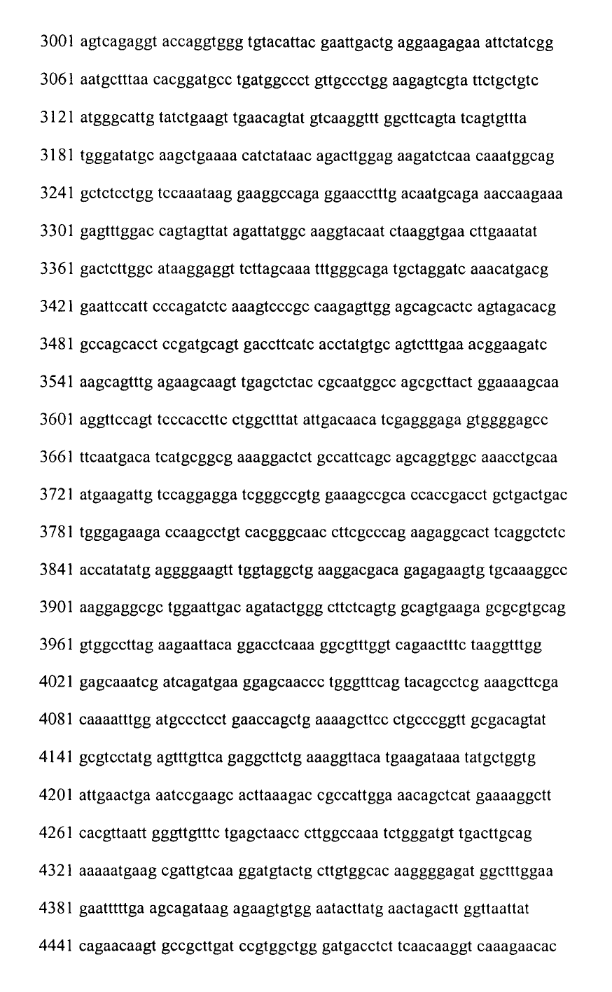

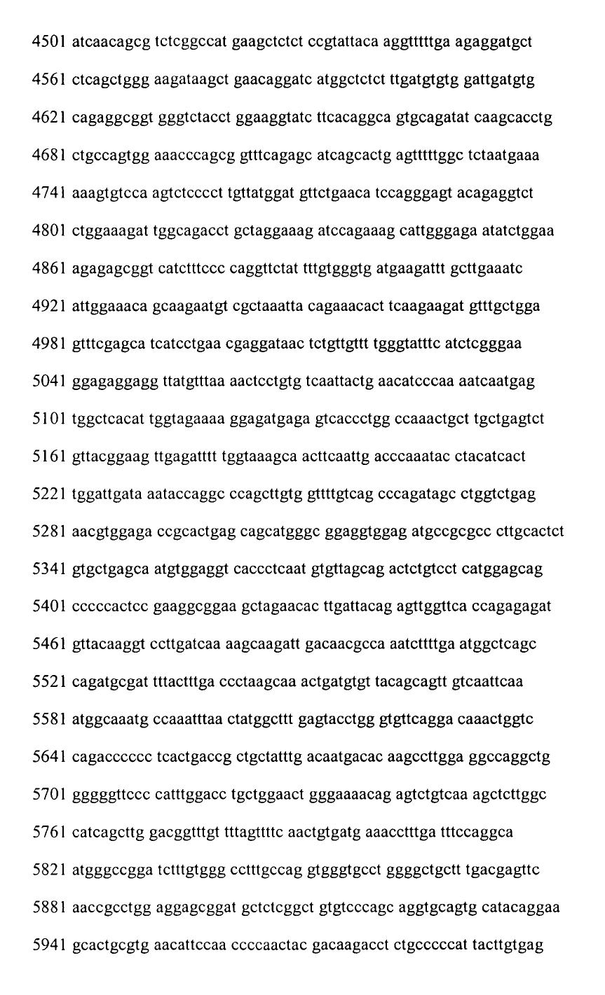

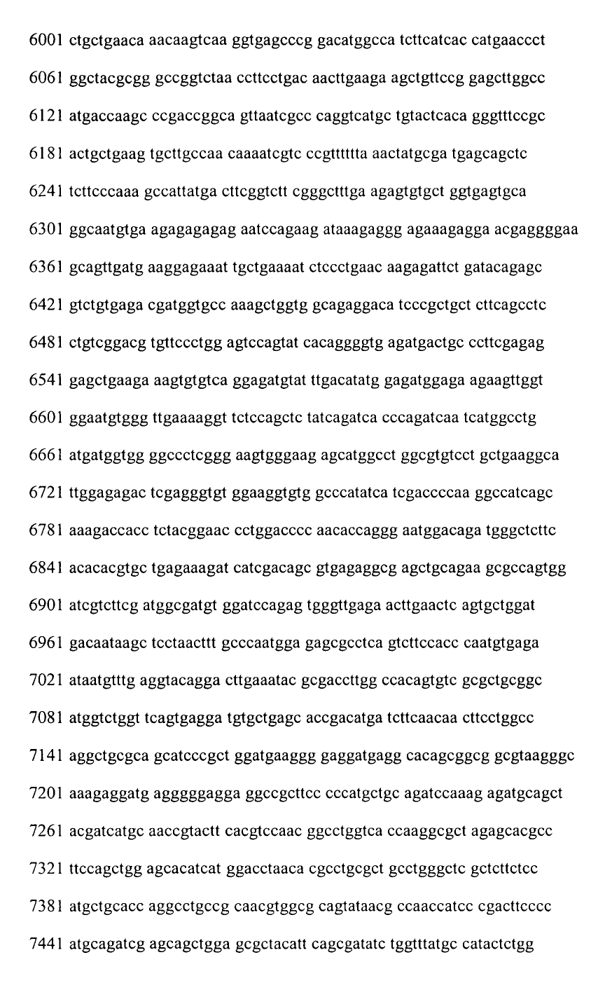

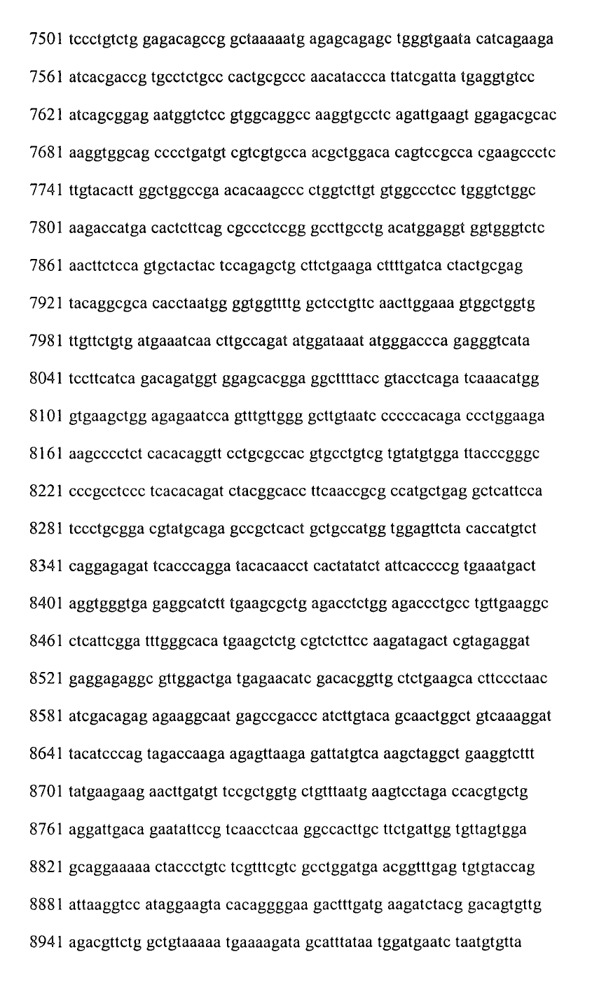

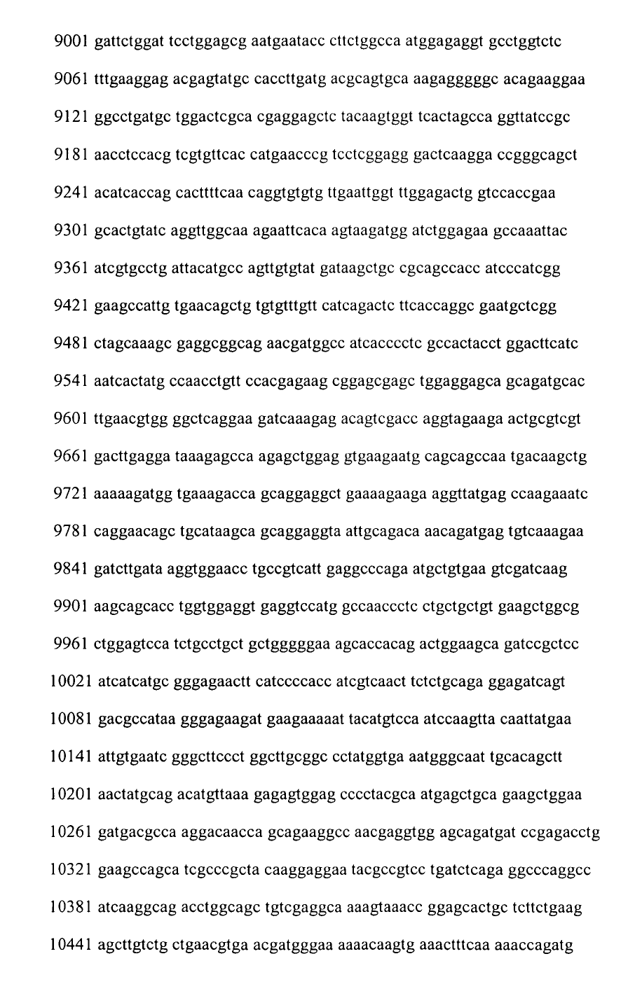

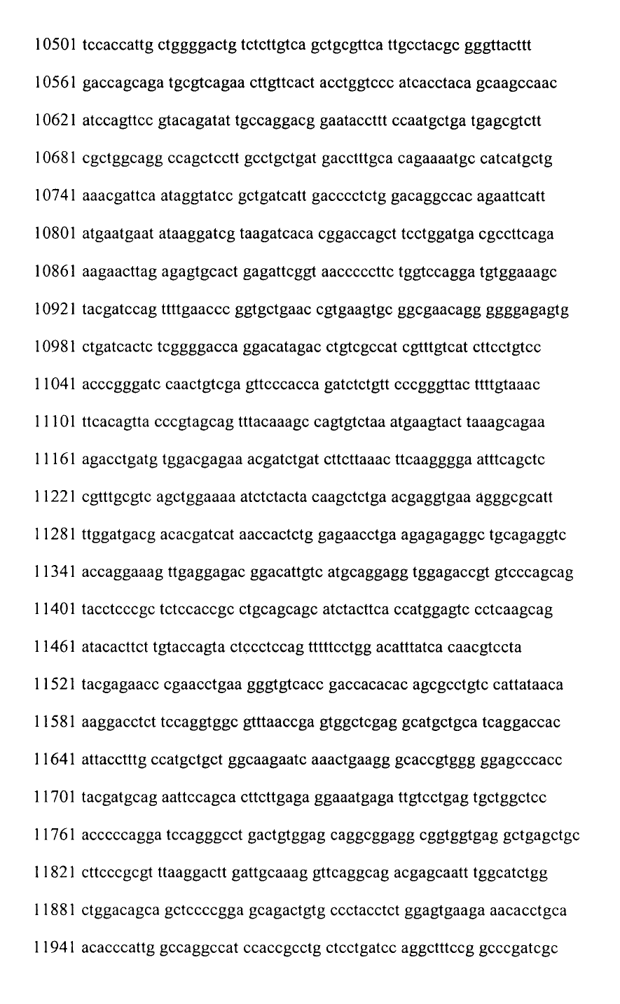

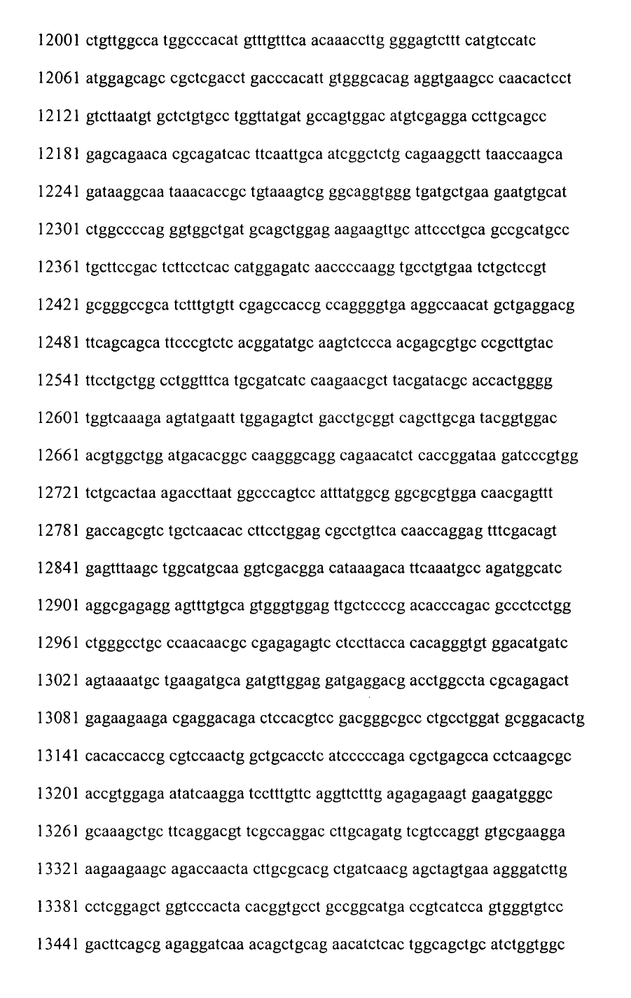

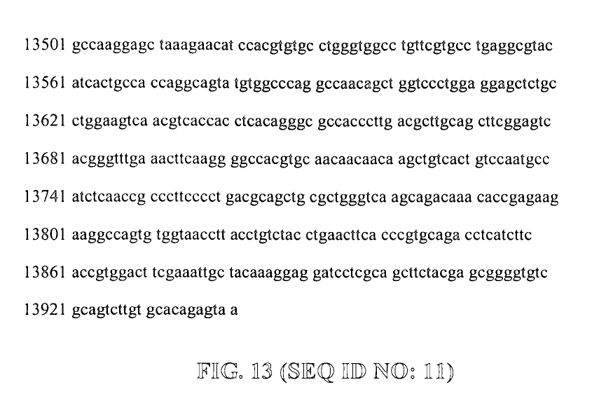

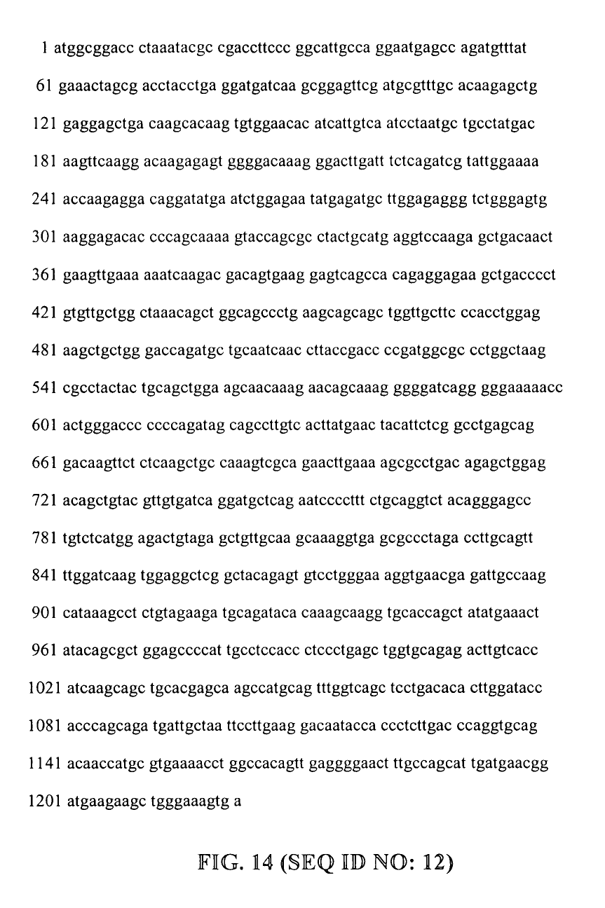

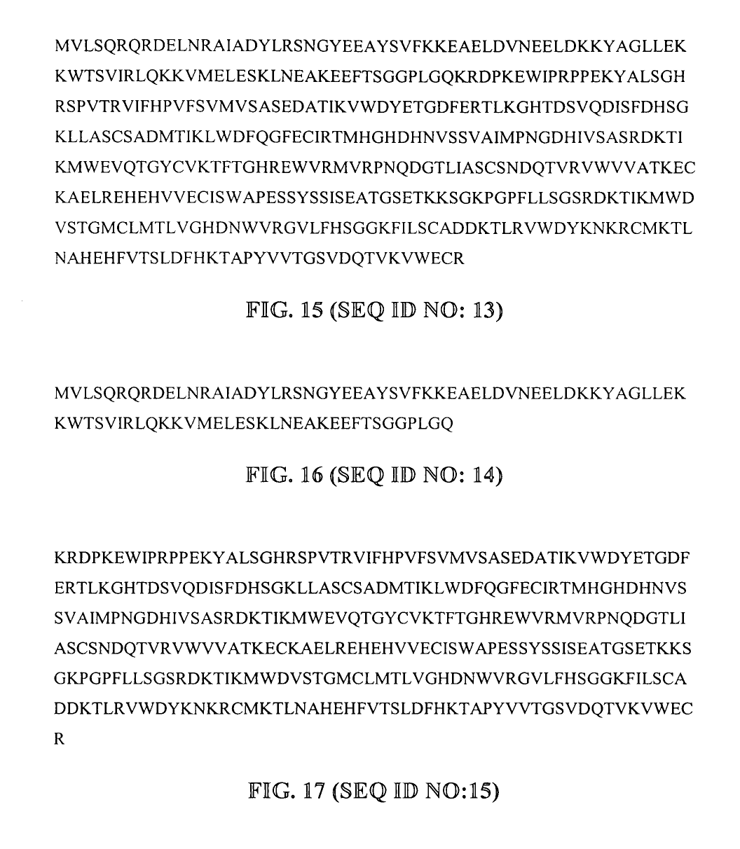

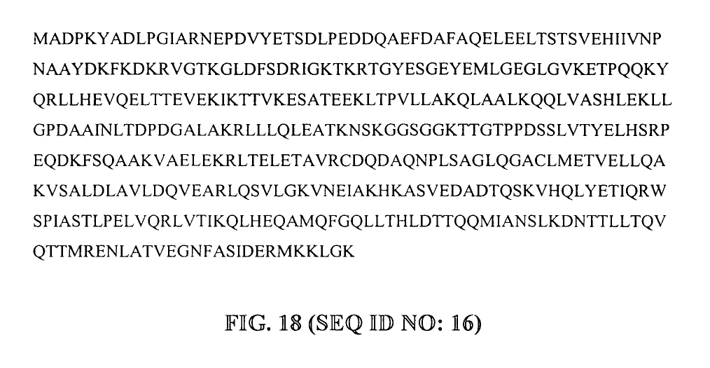

In LIS1 shRNA-transfected brains, multipolar cells again constituted the majority of the cell population by day 2 (71.6±1.8%, n=4 embryos) as they did in control brains. At days 4 and 6, however, most of the cells were not only stalled in the SVZ (FIG. 2 A) but, strikingly, were mostly multipolar (68.0±2.5%, n=5 embryos; and 67.7±2.6%, n=3 embryos, respectively; FIG. 2 B). Only a limited number of bipolar cells were present in the lower IZ at days 2-6 (4.1±0.9%, n=4 embryos; 10.3±1.5%, n=5 embryos; and 10.6±2.3%, n=3 embryos at days 2, 4, and 6, respectively), and limited numbers of cells with radial morphology also persisted throughout (12.0±1.6%, n=4 embryos; 11.4±1.0%, n=5 embryos; and 10.7±1.4%, n=3 embryos at days 2, 4, and 6, respectively). A similar accumulation of cells with a multipolar morphology was observed on days 2 and 4 in brains that were transfected with Cy3-LIS1 siRNA oligonucleotides (FIG. 2 B and FIG. 10). Although cells overexpressing dominant negative cDNA constructs could only be monitored through day 2, a decrease in the percentage of bipolar cells was also observed (GFP control: 11.9±1.5%, n=3; GFP-LIS1N: 4.0±0.8%, n=3; and GFP-LIS1:2.0±0.8%, n=2; FIG. 2 C and FIG. 10). Together, these results suggested that accumulation in the SVZ reflected a block in exit from the multipolar stage of the migratory pathway.

To evaluate the differentiation state of SVZ cells, sections were stained with the neuronal marker TuJ1 (FIG. 11) and progenitor cell markers nestin (FIG. 2 A) and vimentin. Multipolar and bipolar cells that were transfected with LIS1 shRNA, siRNA oligonucleotides, GFP-LIS1N, GFP-LIS1, or GFP-p50 all expressed the neuronal marker TuJ1 but not nestin and vimentin, as is the case for cells transfected with control plasmids or infected with retrovirus (Noctor et al., 2004). These results further demonstrated that cells with reduced LIS1 expression were arrested in a stage corresponding to the multipolar stage of postmitotic neurons in normal brain.

LIS1 has been implicated in cell division by its localization to mitotic kinetochores and the mitotic cell cortex of nonneuronal cells. Dominant negative cDNAs, antibody microinjection, and antisense oligonucleotides all resulted in a pronounced accumulation of cells in prometaphase (Faulkner et al., 2000; Tai et al., 2002). To test for changes in neural progenitor cell cycle progression, brains that were transfected with the empty vector, LIS1 shRNA, or GFP-LIS1N constructs were stained on day 2 using the antiphosphovimentin antibody 4A4, which labels M-phase neural progenitor cells. In control brains that were transfected with empty vector, 3.7±0.9% (n=4) of GFP-expressing cells in the VZ/SVZ were 4A4 positive (FIG. 3, top). Very few LIS1 shRNA- and GFP-LIS1N-transfected cells were 4A4 positive (0.68±0.33%, n=4; and 0.67±0.35%, n=3; respectively). Cells were also immunostained for the nuclear transcription factor Ki67, which is expressed in proliferating cells from S-phase through M-phase of the cell cycle. In day 2 control cells, which were primarily located within the VZ/SVZ, 32.0±4.3% (n=4) of the cells were Ki67 positive (FIG. 3, bottom). In contrast, the percentages of Ki67-positive cells in LIS1 shRNA- (5.4±1.2%, n=4) and GFP-LIS1N (7.2±1.5%, n=3)transfected brains were dramatically decreased (P<0.01).

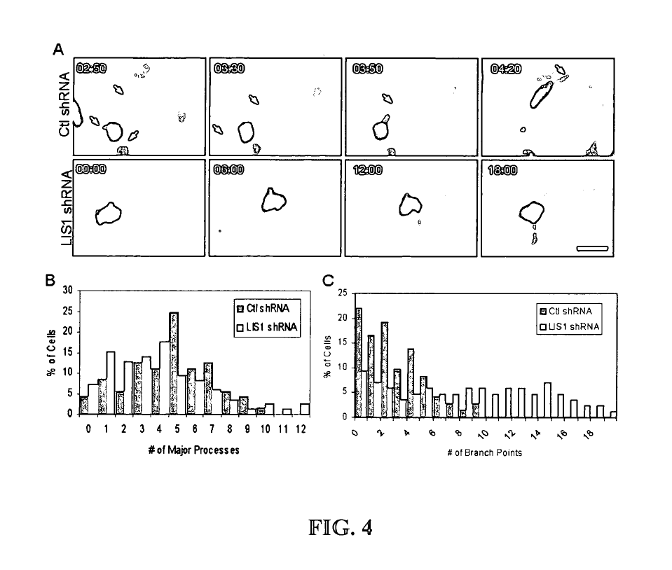

Block in multipolar to bipolar conversion in the SVZ: The mechanisms underlying the conversion of nonmigratory multipolar cells in the SVZ to the bipolar migratory state have not been explored. The accumulation of LIS1-inhibited cells in the multipolar state suggested an important role for LIS1 in this process. To test this possibility directly, slices from brain tissue that had been transfected with LIS1 or control shRNA constructs were placed in culture for live cell imaging. By using a 10-min time-lapse interval during a 10-18-h period from day 2, we detected 14 examples of this morphogenetic event among 73 multipolar cells from 3 brain slices that were examined (FIG. 4). Initially, the cells had multiple processes extending and retracting actively, at which time the cell body appeared relatively immobile. Just before the morphological conversion event, one of the preexisting processes increased in girth and became the leading migratory process as other processes retracted. The newly emergent migratory processes tended to orient toward the pial surface, suggesting an interaction with radial glial fibers, as shown in fixed brain tissues that were stained with nestin and vimentin (FIG. 9). Finally, the cell body followed the direction of the leading process and migrated away. Altogether, the conversion event took only 61±5 min (n=14 cells) to complete.

Almost all of the cells that were transfected with the LIS1 shRNA construct were multipolar and possessed an overall appearance that is similar to that of multipolar control cells (FIG. 4 A). The most dramatic difference was that the transition from the multipolar to the migratory state was completely abolished (0/86 cells in 4 slices), directly demonstrating a block in the migratory pathway at a highly specific stage. In several other regards, experimental and control cells were fairly similar. The number of primary processes (directly protruding from the cell body) was comparable between LIS1 shRNA-transfected cells (3.9±0.3, n=86 cells) and control cells (4.7±0.3, n=73 cells; FIG. 4 B), and the lifetime (119±4 min, n=343 processes) was similar to that of the primary processes of control cells (124±3 min, n=336 processes), indicating that the stability of primary processes was not affected by LIS1 RNAi. However, despite the similarity in overall cell shape, significant differences could be observed. The primary processes in LIS1 shRNA-transfected cells were more branched (8.3±0.7 branch points/cell, n=73 cells) than in control cells (2.6±0.3 branch points/cell, n=86 cells; FIG. 4 C). The branches were usually short (3.5±0.1 μm, n=711 branches) and, more dramatically, as observed in time-lapse video, were far less stable than the primary processes, with an average lifetime of 26±1 min (n=711 branches).

Finally, videos of control and shRNA-transfected cells for the frequency of mitotic events were examined. Consistent with the immunohistochemical analysis, 8/73 control cells divided during time-lapse periods of 12-18 h. In contrast, 0/86 LIS1 shRNA-transfected cells were observed to divide during a comparable period.

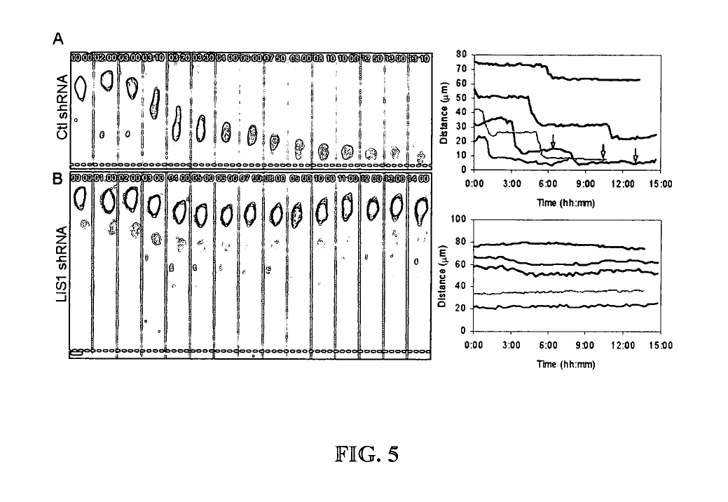

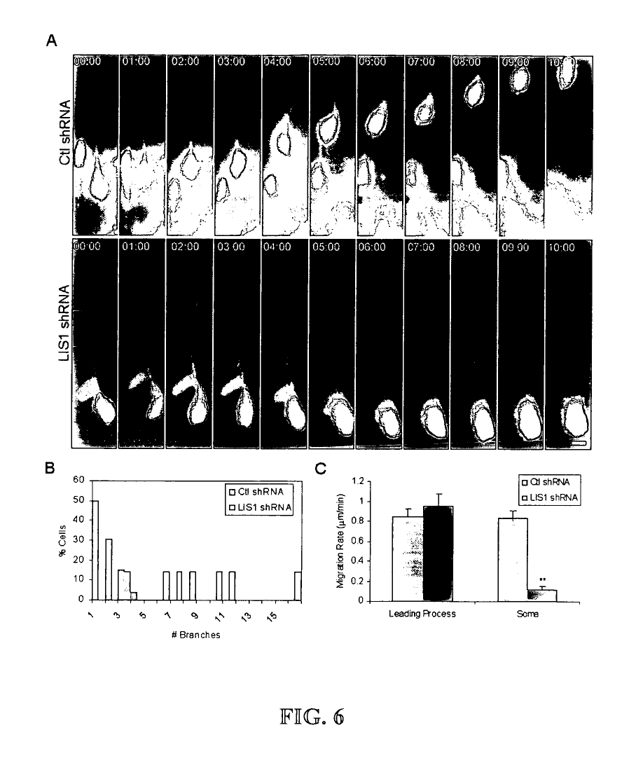

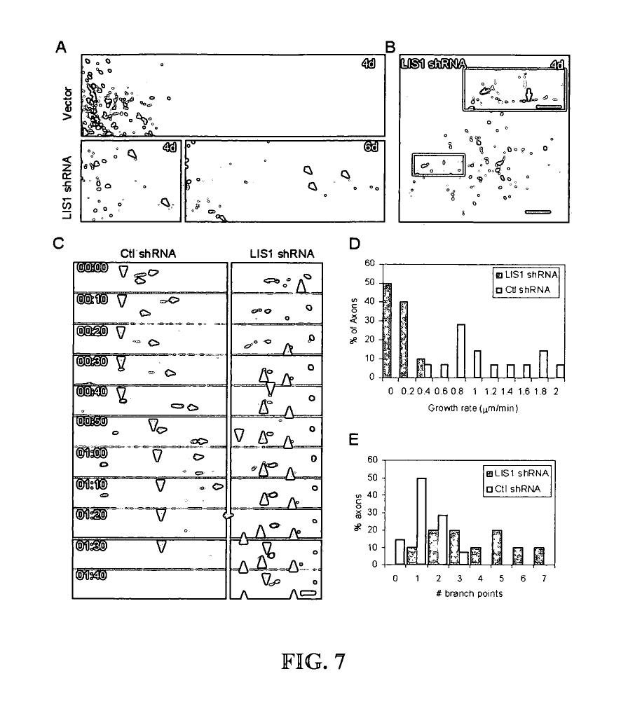

Block in interkinetic nuclear oscillations and cell division in radial glial cells: Despite the accumulation of multipolar cells in the SVZ, a small percentage of radial glial cells remained in the VZ of LIS1 shRNA-transfected brains (FIGS. 1B and 2B). To test whether these cells were still motile, live cell imaging of brain slices was again performed. In control shRNA-transfected cells, nuclei were observed to oscillate, albeit in a discontinuous fashion: periods of immobility were followed by relatively rapid directed movements toward (n=13/28) or away (n=11/28) from the ventricular surface (FIG. 5 A). LIS1 shRNA-transfected radial glial cells appeared to be morphologically normal, exhibiting their characteristic long bipolar radial processes. However, nuclear movement was almost completely abolished among all of the 23 cells from 3 embryos that were monitored. Random movements of very limited range (1-5 μm) were still observed, but directed nuclear migration over substantial distances was eliminated (FIG. 5 B). Nuclei were stalled at various distances from the ventricular surface and maintained their positions over substantial periods of time.

Nuclear division is well established to correlate with position relative to the ventricular surface, although the functional basis for this relationship remains an important mystery in brain development. Among 28 control radial glial cells, 9 were observed to divide during 10-15 h each of direct observation. All divisions occurred at the ventricular surface. In marked contrast, no cell divisions were detected in LIS1 shRNA-transfected radial glial cells (n=0/23 cells in three slices), nor were cells arrested with condensed chromosomes. These results suggest that migration of the nucleus to the ventricular surface is required for radial glial cell division.

Impaired radial migration of bipolar cells: Our results identified two distinct forms of neural progenitor cell behavior that were completely inhibited by LIS1 RNAi. Nonetheless, some cells with a bipolar morphology reached the lower IZ (FIG. 1B). To test whether these cells represented a motile subpopulation of LIS1 shRNA transfectants, live cell imaging was again performed. In control brains, migrating bipolar cells in the IZ were observed between days 3 and 4. The cells extended a leading process toward the CP of relatively constant length (FIG. 6A, top). Movement of the cell bodies was discontinuous as the cell underwent locomotion toward the pial surface.

In LIS1 shRNA-transfected tissue, there were fewer labeled cells (as noted previously) in the IZ (Table 2). The limited number of cells that reached the IZ still had an overall bipolar morphology. However, the soma of all monitored cells from two embryos were completely immobile (FIG. 6 A, bottom). Despite this pronounced inhibitory effect, the leading processes still exhibited active motility. Numerous short and short-lived branches (8.7±1.6 branches/cell, n=7 cells) were observed versus control bipolar cells (1.7±0.2 branches/cell, n=26 cells; FIG. 6 B). More remarkably, process length increased with time (growth rate=0.95±0.12 μm/min, n=7 cells; FIG. 6 C). In control cells, the leading process and soma moved at similar rates (0.85±0.07 and 0.83±0.07 μm, n=26 cells; FIG. 6 C), and, hence, the length of the leading process remained relatively constant (47.6±5.8 μm, n=26 cells; in all cases, cells were recorded before the leading process reached the pial surface). A striking net result was that the leading tip of experimental and control processes extended at almost identical rates (FIG. 6 C). We did not observe branched migration in these cells, as has been reported in studies of p35 mutant (Gupta et al., 2003) and wild-type mice (Tabata and Nakajima, 2003).

Altered axonal extension: In developing neocortex, most migrating neurons put out a trailing process (FIG. 1B, arrows), which is thought to represent the developing axon (Schwartz and Goldman-Rakic, 1991; Noctor et al., 2004). These processes persist as the cells become bipolar and move toward the CP. Despite the accumulation of LIS1 shRNA-transfected cells in the SVZ, tangential axonlike processes were observed at days 4 and 6 (FIG. 7, A and B). These processes extended medially in the same direction as seen for control cells. However, the axonlike processes in LIS1 shRNA-transfected cells were shorter and somewhat curved and branched (FIG. 7 A). The role of LIS1 in this aspect of neural progenitor behavior has not been previously studied. For this reason and to gain further support for the origin and character of these processes, we monitored their behavior in living slices.

In control cells, these processes could be traced back to multipolar SVZ or bipolar IZ cells (FIG. 1 B) and could be observed to extend toward the medial side of the neocortex at a relatively constant rate of 1.19±0.13 μm/min (n=14 axons), which is consistent with their identification as axons (FIG. 7 C). The axons typically had two terminal branches, which alternatively extended and retracted and were tipped by growth cone-like lamellar enlargements. In cells that were transfected with LIS1 shRNA, axons could still be distinguished among the multiple processes of SVZ cells (FIG. 7 C). However, their rate of extension was greatly reduced (0.16±0.04 μm/min; FIG. 7 D), although their orientation was close to that of normal cells. These processes tended to be more highly branched (3.8±0.6 branch points/axon, n=10 axons) than normal (1.3±0.4 branch points/axon, n=14 axons; FIG. 7 E). These branches were very dynamic and had a short average lifetime of 39±4 min (n=38 branches). Importantly, net forward extension of the axons was virtually eliminated.

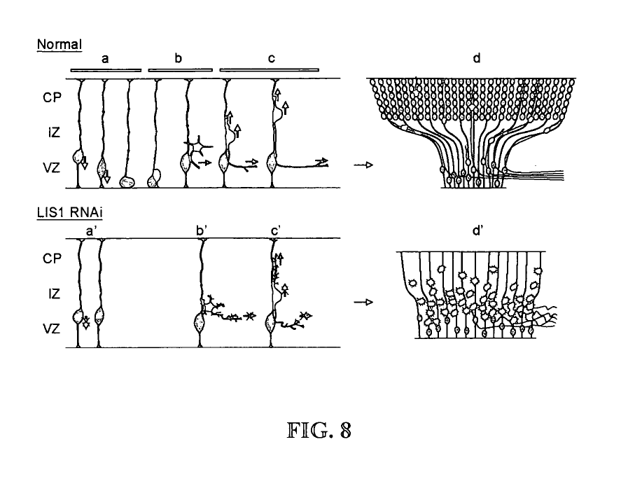

In certain aspects, the present invention provides that acute interference with LIS1 expression or function in a wild-type background dramatically inhibited the redistribution of neural progenitor cells during neocortical development. In certain aspects, LIS1 expression was reduced by RNAi, and overexpression of dominant negative constructs. Live imaging analysis of LIS1 knockdown cells, in certain aspects the invention provides direct evidence for virtually complete arrest at multiple steps of neurogenesis: (1) interkinetic nuclear oscillations within the radial glial cells and the attendant mitotic divisions; (2) conversion of multipolar SVZ cells to the bipolar migratory state; (3) the locomotion of bipolar cells through the IZ; and (4) the axonal growth of migratory neurons (FIG. 8). This pattern of effects is inconsistent with a single general effect on neural progenitor cell maturation or migration. It is more readily explained as the result of distinct effects at a number of different stages in the migratory and cell cycle pathways. It is likely that this outcome is a result of uptake of the shRNA-expressing vector at a range of concentrations and cell cycle stages by neural progenitor cells that are exposed at the ventricular surface. This pattern of inhibition appears to provide a unique means to gain direct insight into the complete range of neuronal functions in which LIS1 participates. Each of the observed effects, in turn, provides new insight into the etiology of lissencephaly.

LIS1 is essential for interkinetic nuclear oscillation and entry into cell division: The earliest aspect of neurogenesis in which LIS1 is implicated by our data are the interkinetic nuclear oscillations of neural progenitor cells within the VZ (FIG. 5), which have only recently been identified as radial glial cells (Malatesta et al., 2000; Miyata et al., 2001; Noctor et al., 2001). In control radial glial cells, somal movements were saltatory but mostly unidirectional during the substantial periods of continuous observation that were involved in our experiments (FIG. 5). LIS1 RNAi completely abolished directed somal motility. This result represents the first direct demonstration that LIS1 is involved in the classic interkinetic nuclear oscillations of mammalian neural progenitor cells. It supports the hypothesis that vertebrate LIS1, like its fungal counterpart NudF (Xiang et al., 1995), is involved in nuclear translocation within cell processes. Thus, dynein which is regulated by LIS1, is the force generator for nuclear movement in radial glial progenitors.

A long-standing issue in the field of brain development is the functional significance of interkinetic nuclear oscillations, and whether nuclear position merely correlates with cell cycle stage or whether it controls it. In certain aspects, the invention provides that there were no mitotic events in cells expressing LIS1 shRNA, which is in dramatic support of the latter possibility. This evidence could relate to the defects in cell division resulting from the expression of dominant negative cDNAs, antibody injection, and antisense oligonucleotides in our previous work (Faulkner et al., 2000; Tai et al., 2002). However, each of these treatments caused cells to accumulate in prometaphase with a markedly increased mitotic index overall. This contrasts dramatically with the current situation in which no mitotic events were observed in VZ and SVZ progenitor cells, and mitotic index was substantially reduced.

In the case of radial glial cells in the VZ, an appealing possibility is that known or unknown mitogenic factors that are enriched at the ventricular surface are required not only in controlling neurogenesis but also in controlling mitotic entry. In this case, the inability of nuclei to reach the ventricular surface in our experiments would limit their access to mitogenic agents. This possibility is supported by a decrease in the percentage of LIS1 shRNA-transfected radial glial cells that are positive for phosphovimentin or that exhibit condensed chromosomes as judged by DAPI staining. The few mitotic cells seen in the latter analysis were all located at the ventricular surface. These may represent rare cells, not detected by the live cell imaging, which retained sufficient LIS1 to enter mitosis. However, their exclusive location at the ventricular surface further supports a spatial control mechanism for mitotic entry.

A reduced number of mitotic figures was observed within the SVZ in fixed brain tissue and in the number of Ki67-positive multipolar cells (FIG. 3). Thus, it appears that the number of SVZ intermediate progenitor cells, which are still capable of division (Noctor et al., 2004), was significantly reduced by interference with LIS1 expression or function. The reduced numbers of mitotic cells that were observed within the VZ and SVZ can result from related or different mechanisms. It is likely that those transfected cells that reach the SVZ cells must, nonetheless, have experienced some effect of reduced LIS1 expression (e.g., a slower journey to their final destination). Conceivably, such a delay could alter fate determination for these cells, allowing premature exit from the cell cycle.

In a previous study with an LIS1 heterozygote mouse, cell divisions were reported to increase in the SVZ but decrease in the VZ (Gambello et al., 2003). Although these divisions were said to be in part “ectopic,” it was not possible to distinguish whether they involved radial glial or SVZ intermediate progenitor cells. These results, therefore, cannot be readily related to the present study. Based on the current and previous results, it is likely that cell division should proceed under conditions of haploinsufficiency, albeit at a reduced frequency. Division itself, however, should be prolonged. The net result may have different effects on mitotic index in the VZ and SVZ, but the precise quantitative outcome is difficult to predict. Increased ectopic divisions were recently reported in mice that were heterozygous null for NudE, which is another protein in the LIS1 and dynein pathway (Feng and Walsh, 2004). Thus, as predicted from in vitro studies (Faulkner et al., 2000), brain developmental disorders arising from mutations in the dynein pathway now appear to involve defects in cell proliferation as well as in migration.

LIS1 is necessary for progression from the multipolar to the migratory stage: The most striking effect of LIS1 RNAi and dominant negative LIS1 and dynactin cDNAs on the overall distribution of transfected neural progenitors was the accumulation of cells within the SVZ (FIG. 2), as was previously observed in RNAi analysis of the lissencephaly genes doublecortin (LoTurco, 2004) and LIS1 (Shu et al., 2004). Whether this pattern reflected a general migration delay or interference with specific steps in the migratory pathway was unknown. The results presented herein demonstrate defect in specific steps in the migratory pathway. Although the previous study involving the use of LIS1 RNAi reported the bipolar morphology to be predominant in the SVZ (Shu et al., 2004), the results presented herein using both static and live cell imaging showed an absolute block in progression from the multipolar to the migratory stage (FIG. 4).

A specific block in exit from the SVZ is of considerable interest in understanding lissencephaly. This condition is characterized by the presence of a broad ectopic neuronal lamina within the white matter of the newborn neocortex (Dobyns and Truwit, 1995). Based on the results disclosed herein it is likely that this and, perhaps, other lamination defects reflect changes in specific rate-limiting steps in the neuronal migration pathway, predominantly involving exit from the SVZ.

The specific role of LIS1 in the conversion of multipolar to bipolar cells is uncertain. The first detectable event in this process was a thickening of the presumptive migratory process, which the nucleus subsequently enters (FIG. 4). This observation suggests that LIS1 and its regulatory target dynein are involved in the shift in cytoplasmic contents into the differentiating migratory process. Previously it was observed that dynein and LIS1 are associated with the leading edge of migrating fibroblasts (Dujardin et al., 2003). Als, an accumulation of dynein and LIS1 at the tips of rapidly growing laminin-induced neurites was observed in primary chick dorsal root ganglia neurons. It is likely that LIS1 and dynein play a related role in the initiation of the migratory process, although, not surprisingly, in its subsequent growth. The results presented herein suggest that at the reduced levels attained in this study, LIS1 is not required for entry into the multipolar state or in process maintenance.

Uncoupling of nucleokinesis from migratory process growth: The number of LIS1 siRNA-, shRNA-, and dominant negative LIS1- and dynactin cDNA-transfected cells within the IZ and CP were greatly reduced relative to controls, which is consistent with a small probability of escape from the SVZ (FIG. 1). Those few cells that reached this region were not yet connected with the pial surface of the developing brain. Migration should, therefore, occur by locomotion (Nadarajah et al., 2001), which involves forward extension of the migratory process and somal translocation to keep up. The shRNA-expressing cells had a bipolar morphology, which is comparable in low magnification images with control cells. However, migration in shRNA-expressing cells was completely abolished.

In striking contrast to normal bipolar cells, however, the soma were virtually immotile (FIG. 6). Nonetheless, growth of the migratory process persisted. Together, these results provide the first indication that these aspects of locomotion can be uncoupled. As is the case for SVZ cells, the migratory processes of LIS1 shRNA-transfected IZ cells exhibited numerous short, highly dynamic extensions. Whether these represent branches that are similar to those observed in axons or whether they are more similar to filopodial extensions has to be determined. Net axonal growth stopped in LIS1 knockdown cells, whereas the leading migratory processes of bipolar cells maintained their ability to extend. One possible explanation for these differences is that the migratory process, but not the axon, is guided and supported by radial glial fibers, where dynein and LIS1 might be less essential. The results presented herein indicated that the basic roles of dynein and LIS1 may differ between process types.

Other studies have found that reduced expression of LIS1 and its interacting proteins NudE and NudEL or dynein inhibited somal translocation in dissociated neuronal cell cultures (Hirotsune et al., 1998; Gambello et al., 2003; Shu et al., 2004; Tanaka et al., 2004). Clear effects on somal movement were observed, but it is uncertain whether they represent nuclear migration within processes or traction-mediated translocation of the entire somal region. Recent evidence has suggested a role for LIS1, dynein, and NudEL in generating tension between the nucleus and centrosome in migratory neural progenitor cells (Shu et al., 2004; Tanaka et al., 2004), which could contribute to somal translocation (Solecki et al., 2004). Based on evidence from other systems, this behavior is likely to be mediated by cortically associated dynein and LIS1, with which centrosome-tethered microtubules interact (Palazzo et al., 2001; Dujardin et al., 2003; Etienne-Manneville and Hall, 2001). It is likely that the observed defect in somal movement within bipolar IZ cells that were transfected with LIS1 shRNA may, in part, involve a similar pool of cortical dynein that is unable to engage cytoplasmic microtubules.

LIS1 is important in axonal extension: A single long axon was detected extending from both multipolar and bipolar cells based on outgrowth length, caliber, and direction of the process. The axon remained clearly identifiable in cells that were subjected to LIS1 RNAi, demonstrating the persistence of underlying polarity in multipolar and bipolar cells. The persistence of axons, despite clear interference with their continued extension, may indicate that growth was initiated before LIS1 levels had become severely reduced. These considerations suggest that LIS1 is not required for maintenance of processes once they have formed.

How LIS1 and dynein function to control process dynamics has been the subject of investigation. Inhibition of cytoplasmic dynein function using dynamitin was reported to induce myosin-mediated neurite retraction (Ahmad et al., 2000). Also, LIS1 and dynein become concentrated at sites of nascent process formation at the leading edge of chick sympathetic and dorsal root ganglia growth cones in response to laminin and that they persist at the tips of rapidly growing processes. Acute interference with either LIS1 or dynein by antibody injection eliminated growth cone remodeling. Similar effects occur in the axons of SVZ cells that were transfected with LIS1 shRNA. The results presented herein suggest that elongation of the two types of process is controlled by substantially different mechanisms.

The function of early appearing axonal processes characterized here is not well established, and the effects that were observed on their extension had not been anticipated from prior studies. Use of retrograde tracing has shown connectivity of migrating neurons in the fetal monkey cerebrum to the opposite cerebral hemisphere (Schwartz and Goldman-Rakic, 1991). The processes that were observed (FIG. 7) exhibited organized and directed growth toward the midline of the cerebrum, strongly suggesting that they correspond to those involved in connectivity to other brain regions. The truncated axons that result from reduced LIS1 expression seem unlikely to be capable of reaching their targets. These results, therefore, raise the possibility that similar, albeit less pronounced, effects may be involved in lissencephaly. Previous studies in hippocampus of LIS1 heterozygous mice and fruit flies showed stunted dendritic branches and abnormal synaptic transmission (Fleck et al., 2000; Liu et al., 2000). The effects on axon outgrowth that were identified here could well result in connectivity defects (Ross, 2002). These, in turn, may have severe consequences for brain function and could contribute to the loss of higher brain function, the frequency of epilepsy, cerebral palsy, and seizures that are characteristic of classical lissencephaly, and dramatically decreased life span.

Human glioblastoma cell lines: U87 MG, T98MG, U251, will be purchased from American Type Culture Collection and cultured in DMEM supplemented with 10% FBS (Intergen Co., Purchase, N.Y., USA), penicillin (1OOIU/mL), and streptomycin (10OIlg/mL) (Invitrogen Life Technologies, Carlsbad, Calif., USA) at 37° C. and 5% CO2.

LIS1 inhibition by iRNA in glioma cells: 10⁶ U87 cells will be plated and incubated overnight. The cells are then infected with vector pRNAT-LIS1 expressing shRNA of SEQ ID NO: 1, or a control vector at a multiplicity of infection (MOI) of 100 for 3 h at 37° C. Protein levels of LIS1 will be measured by Western blot to demonstrate that LIS1 is inhibited by the shRNA of SEQ ID NO: 1. Cell division, proliferation and migration of glioblastoma cells are expected to be significantly reduced compared to glioblastoma cells transfected with a control vector.

LIS1 inhibition in vivo: U87 cells (5×10⁶) in DMEM will be injected subcutaneously (se) into the right hind-limb of nude mice. When tumors reach an average size of 200 mm3 (length×width×thickness/2), the tumor-bearing mice will be divided into 2 groups: 1. Untreated Control (UTC); 2. LIS1 shRNA of SEQ ID NO: 1, expressed by vector pRNAT-LIS1. Vector pRNAT-LIS1 will be injected intratumorally (IT) at a dose of 2×10⁸ particle units (pu) each day. Two consecutive daily injections are given, control animals received serum free medium (SFM). In one part of the study, animals will be euthanized on day 2 and 4 (i.e., 48 h and 96 h after treatment initiation), tumors harvested, snap-frozen in liquid nitrogen and homogenized in RIPA buffer (150 mM NaCl, 10 mM Tris at pH 7.5, 5 mM EDTA at pH 7.5, 100 mM PMSF, 1 μg/mL leupeptin, and 2 μg/mL aprotinin). LIS1 protein level will be measured be western blot, and is expected to be reduced compared to the control animals. In another part of the study, tumor volume is measured every 2-3 days. Fractional tumor volume (V/Vo where V0=volume on day 0) is calculated and plotted. Tumor volume is expected to become reduced in the shRNA treated animals compared to the control animals.

U87 cell viability studies: Trypan Blue dye exclusion method is employed. 10⁴ U87 cells will be plated and incubated at 37° C. overnight. Subsequently, the cells will be treated with antagonist of LIS1, dynein or dynactin, and incubated for 3 h, and washed. At 24 h, 48 h, and 72 h following exposure to agent, the cells are trypsinized and the viable cell number/well determined using a hemocytometer. Cell viability at 72 h will be verified using the MTS calorimetric assay, per the manufacturer's protocol (Cell Titer 96 Aqueous, One Solution cell proliferation assay, Promega Corporation, Madison, Wis., USA). Optical density was read at 490 nm using an ELISA microplate reader after 1.5 h, at 37° C.

Anti-LIS1 therapy against tumor cells implanted into the brains of immunocompromised animals: 5×10⁵ U87 cells will be inoculated into the right caudate nucleus on day 0 using a screw guide technique (Lal, S. et al. (2000) JNeurosurg 92:326-333) of nude mice or rats, and the tumor growth and the effect of anti-LIS1 therapy will bw assessed. Anti-LIS1 therapy in this example will include intracranial administration of the pRNAT-LIS1 expressing shRNA of SEQ ID NO: 1, or the siRNA of SE ID NO: 2, or the iRNA of SEQ ID NO: 5. Implantation of the cells into brains rather than subcutaneously provides an environment closer to the tissue of origin of the glioma cells. At different stages of growth after the xenograft implantation, tumor and surrounding tissues will be collected and histopathology studies are carried out to examine the degree of tumor invasion, vascularization, production of angiogenic factors and the expression of LIS1 in tumor cells. All of these methods are conventional. The inhibition of LIS1 activity by the shRNAs is expected to abrogate growth and other malignant properties of the implanted cells. Such experiments are corroborated with studies of freshly excised as well as preserved human glioma specimens. The latter experiments are expected to validate the potential of shRNAs against LIS1 as a treatment for glioma progression.

TABLE 1
Summary of animals and cells used in still image analyses

| | | Number | Total | | | |
|---|---|---|---|---|---|---|
| | | of | Number | Percentage of Cells within | | |
| Treatment | Age | Animals | of Cells | VZ/SVZ | IZ | CP |
| LIS1 shRNA | E16/18 | 4 | 1289 | 88.2% | 9.8% | 2.1% |
| | E16/20 | 5 | 1764 | 84.9% | 10.4% | 4.7% |
| | E16/22 | 3 | 755 | 80.1% | 16.2% | 3.7% |
| LIS1-3 | E16/18 | 3 | 1163 | 86.5% | 12.4% | 1.0% |
| mtshRNA | E16/20 | 3 | 1649 | 26.7% | 24.6% | 48.7% |
| | E16/22 | 3 | 937 | 7.2% | 17.5% | 75.3% |
| Empty | E16/18 | 4 | 1650 | 85.2% | 11.8% | 3.0% |
| vector | E16/20 | 5 | 3021 | 21.4% | 25.5% | 53.1% |
| | E16/22 | 3 | 1074 | 3.8% | 7.1% | 89.0% |
| LISI-siRNA | E16/18 | 3 | 412 | 87.6% | 7.8% | 4.5% |
| oligo | E16/20 | 2 | 121 | 83.4% | 7.8% | 8.9% |
| | E16/22 | 8 | — | — | — | — |
| GFP-LIS1N | E16/18 | 3 | 1043 | 91.6% | 7.9% | 0.5% |
| | E16/20 | 12 | — | — | — | — |
| GFP-LIS1 | E16/18 | 2 | 658 | 93.1% | 6.8% | 0.2% |
| | E16/20 | 11 | — | — | — | — |
| GFP-p50 | E16/18 | 3 | 396 | 96.5% | 3.4% | 0.1% |
| | E16/20 | 6 | — | — | — | — |
| GFP | E16/18 | 3 | 1362 | 87.5% | 10.4% | 2.1% |
| | E16/20 | 3 | 1727 | 22.8% | 27.1% | 50.1% |

| | | |
|---|---|---|
| Total | 89 | 19021 |

TABLE 2
Summary of animals and cells used in live cell analyses

| | | | Total | |
|---|---|---|---|---|
| | | Number of | Number of | Area of |
| Treatment | Age | Animals | Cells | Interest |
| LIS1 shRNA | E16/18 | 4 | 86 | SVZ |
| | E16/18 | 2 | 10 | Axon |
| | E16/17.5 | 3 | 23 | VZ |
| | E16/19 | 3 | 7 | IZ |
| LIS1-3mtshRNA | E16/18 | 3 | 73 | SVZ |
| | E16/18 | 2 | 14 | Axon |
| | E16/17.5 | 3 | 28 | VZ |
| | E16/19 | 2 | 26 | IZ |

| | | |
|---|---|---|
| Total | 22 | 267 |

### REFERENCES

Ahmad, F. J., J. Hughey, T. Wittmann, A. Hyman, M. Greaser, and P. W. Baas. 2000. Motor proteins regulate force interactions between microtubules and microfilaments in the axon. Nat. Cell Biol. 2:276-280.
Cahana, A., T. Escamez, R. S. Nowakowski, N. L. Hayes, M. Giacobini, A. von Holst, O. Shmueli, T. Sapir, S. K. McConnell, W. Wurst, et al. 2001. Targeted mutagenesis of Lis1 disrupts cortical development and LIS1 homodimerization. Proc. Natl. Acad. Sci. USA. 98:6429-6434.
Dobyns, W. B., and C. L. Truwit. 1995. Lissencephaly and other malformations of cortical development: 1995 update. Neuropediatrics. 26:132-147.
Dujardin, D. L., L. E. Barnhart, S. A. Stehman, E. R. Gomes, G. G. Gundersen, and R. B. Vallee. 2003. A role for cytoplasmic dynein and LIS1 in directed cell movement. J. Cell Biol. 163:1205-1211.
Echeverri, C. J., B. M. Paschal, K. T. Vaughan, and R. B. Vallee. 1996. Molecular characterization of the 50-kD subunit of dynactin reveals function for the complex in chromosome alignment and spindle organization during mitosis. J. Cell Biol. 132:617-633.
Efimov, V. P., and N. R. Morris. 2000. The LIS1-related NUDF protein of Aspergillus nidulans interacts with the coiled-coil domain of the NUDE/RO11 protein. J. Cell Biol. 150:681-688.
Etienne-Manneville, S., and A. Hall. 2001. Integrin-mediated activation of Cdc42 controls cell polarity in migrating astrocytes through PKCzeta. Cell. 106:489-498.
Faulkner, N. E., D. L. Dujardin, C. Y. Tai, K. T. Vaughan, C. B. O'Connell, Y. Wang, and R. B. Vallee. 2000. A role for the lissencephaly gene LIS1 in mitosis and cytoplasmic dynein function. Nat. Cell Biol. 2:784-791.
Feng, Y., and C. A. Walsh. 2004. Mitotic spindle regulation by Nde1 controls cerebral cortical size. Neuron. 44:279-293.
Fleck, M. W., S. Hirotsune, M. J. Gambello, E. Phillips-Tansey, G. Suares, R. F. Mervis, A. Wynshaw-Boris, and C. J. McBain. 2000. Hippocampal abnormalities and enhanced excitability in a murine model of human lissencephaly. J. Neurosci. 20:2439-2450.
Gambello, M. J., D. L. Darling, J. Yingling, T. Tanaka, J. G. Gleeson, and A. Wynshaw-Boris. 2003. Multiple dose-dependent effects of Lis1 on cerebral cortical development. J. Neurosci. 23:1719-1729.
Geiser, J. R., E. J. Schott, T. J. Kingsbury, N. B. Cole, L. J. Totis, G. Bhattacharyya, L. He, and M. A. Hoyt. 1997. Saccharomyces cerevisiae genes required in the absence of the CIN8-encoded spindle motor act in functionally diverse mitotic pathways. Mol. Biol. Cell. 8:1035-1050.
Gupta, A., K. Sanada, D. T. Miyamoto, S. Rovelstad, B. Nadarajah, A. L. Pearlman, J. Brunstrom, and L. H. Tsai. 2003. Layering defect in p35 deficiency is linked to improper neuronal-glial interaction in radial migration. Nat. Neurosci. 6:1284-1291.
Hattori, M., H. Adachi, M. Tsujimoto, H. Arai, and K. Inoue. 1994. Miller-Dieker lissencephaly gene encodes a subunit of brain platelet-activating factor acetylhydrolase. Nature. 370:216-218 (corrected).
Haubensak, W., A. Attardo, W. Denk, and W. B. Huttner. 2004. Neurons arise in the basal neuroepithelium of the early mammalian telencephalon: a major site of neurogenesis. Proc. Natl. Acad. Sci. USA. 101:3196-3201.
Hirotsune, S., M. W. Fleck, M. J. Gambello, G. J. Bix, A. Chen, G. D. Clark, D. H. Ledbetter, C. J. McBain, and A. Wynshaw-Boris. 1998. Graded reduction of Pafah1b1 (Lis1) activity results in neuronal migration defects and early embryonic lethality. Nat. Genet. 19:333-339.
Kholmanskikh, S. S., J. S. Dobrin, A. Wynshaw-Boris, P. C. Letoumeau, and M. E. Ross. 2003. Disregulated RhoGTPases and actin cytoskeleton contribute to the migration defect in Lis1-deficient neurons. J. Neurosci. 23:8673-8681.
Kitagawa, M., M. Umezu, J. Aoki, H. Koizumi, H. Arai, and K. Inoue. 2000. Direct association of LIS1, the lissencephaly gene product, with a mammalian homologue of a fungal nuclear distribution protein, rNUDE. FEBS Lett. 479:57-62.
Koizumi, H., N. Yamaguchi, M. Hattori, T. O. Ishikawa, J. Aoki, M. M. Taketo, K. Inoue, and H. Arai. 2003. Targeted disruption of intracellular type I platelet activating factor-acetylhydrolase catalytic subunits causes severe impairment in spermatogenesis. J. Biol. Chem. 278:12489-12494.
Liu, Z., R. Steward, and L. Luo. 2000. Drosophila Lis1 is required for neuroblast proliferation, dendritic elaboration and axonal transport. Nat. Cell Biol. 2:776-783.
Lo Nigro, C., C. S. Chong, A. C. Smith, W. B. Dobyns, R. Carrozzo, and D. H. Ledbetter. 1997. Point mutations and an intragenic deletion in LIS1, the lissencephaly causative gene in isolated lissencephaly sequence and Miller-Dieker syndrome. Hum. Mol. Genet. 6:157-164.
LoTurco, J. 2004. Doublecortin and a tale of two serines. Neuron. 41:175-177.
Malatesta, P., E. Hartfuss, and M. Gotz. 2000. Isolation of radial glial cells by fluorescent-activated cell sorting reveals a neuronal lineage. Development. 127:5253-5263.
Misson, J. P., C. P. Austin, T. Takahashi, C. L. Cepko, and V. S. Caviness Jr. 1991. The alignment of migrating neural cells in relation to the murine neopallial radial glial fiber system. Cereb. Cortex. 1:221-229.
Miyata, T., A. Kawaguchi, H. Okano, and M. Ogawa. 2001. Asymmetric inheritance of radial glial fibers by cortical neurons. Neuron. 31:727-741.
Mizuguchi, M., S. Takashima, A. Kakita, M. Yamada, and K. Ikeda. 1995. Lissencephaly gene product. Localization in the central nervous system and loss of immunoreactivity in Miller-Dieker syndrome. Am. J. Pathol. 147:1142-1151.
Morris, S. M., U. Albrecht, O. Reiner, G. Eichele, and L. Y. Yu-Lee. 1998. The lissencephaly gene product Lis1, a protein involved in neuronal migration, interacts with a nuclear movement protein, NudC. Curr. Biol. 8:603-606.
Nadarajah, B., J. E. Brunstrom, J. Grutzendler, R. O. Wong, and A. L. Pearlman. 2001. Two modes of radial migration in early development of the cerebral cortex. Nat. Neurosci. 4:143-150.
Niethammer, M., D. S. Smith, R. Ayala, J. Peng, J. Ko, M. S. Lee, M. Morabito, and L. H. Tsai. 2000. NUDEL is a novel Cdk5 substrate that associates with LIS1 and cytoplasmic dynein. Neuron. 28:697-711.
Noctor, S. C., A. C. Flint, T. A. Weissman, R. S. Dammerman, and A. R. Kriegstein. 2001. Neurons derived from radial glial cells establish radial units in neocortex. Nature. 409:714-720.
Noctor, S. C., V. Martinez-Cerdeno, L. Ivic, and A. R. Kriegstein. 2004. Cortical neurons arise in symmetric and asymmetric division zones and migrate through specific phases. Nat. Neurosci. 7:136-144.
Palazzo, A. F., H. L. Joseph, Y. J. Chen, D. L. Dujardin, A. S. Alberts, K. K. Pfister, R. B. Vallee, and G. G. Gundersen. 2001. Cdc42, dynein, and dynactin regulate MTOC reorientation independent of Rho-regulated microtubule stabilization. Curr. Biol. 11:1536-1541.
Rakic, P., L. J. Stensas, E. Sayre, and R. L. Sidman. 1974. Computer-aided three-dimensional reconstruction and quantitative analysis of cells from serial electron microscopic montages of foetal monkey brain. Nature. 250:31-34.
Reiner, O., R. Carrozzo, Y. Shen, M. Wehnert, F. Faustinella, W. B. Dobyns, C. T. Caskey, and D. H. Ledbetter. 1993. Isolation of a Miller-Dieker lissencephaly gene containing G protein beta-subunit-like repeats. Nature. 364:717-721.
Ross, M. E. 2002. Brain malformations, epilepsy, and infantile spasms. Int. Rev. Neurobiol. 49:333-352.
Saito, T., and N. Nakatsuji. 2001. Efficient gene transfer into the embryonic mouse brain using in vivo electroporation. Dev. Biol. 240:237-246.
Sasaki, S., A. Shionoya, M. Ishida, M. J. Gambello, J. Yingling, A. Wynshaw-Boris, and S. Hirotsune. 2000. A LIS1/NUDEL/cytoplasmic dynein heavy chain complex in the developing and adult nervous system. Neuron. 28:681-696.
Schwartz, M. L., and P. S. Goldman-Rakic. 1991. Prenatal specification of callosal connections in rhesus monkey. J. Comp. Neurol. 307:144-162.
Shu, T., R. Ayala, M. D. Nguyen, Z. Xie, J. G. Gleeson, and L. H. Tsai. 2004. Ndell operates in a common pathway with LIS1 and cytoplasmic dynein to regulate cortical neuronal positioning. Neuron. 44:263-277.
Smith, D. S., M. Niethammer, R. Ayala, Y. Zhou, M. J. Gambello, A. Wynshaw-Boris, and L. H. Tsai. 2000. Regulation of cytoplasmic dynein behaviour and microtubule organization by mammalian Lis1. Nat. Cell Biol. 2:767-775.
Solecki, D. J., L. Model, J. Gaetz, T. M. Kapoor, and M. E. Hatten. 2004. Par6alpha signaling controls glial-guided neuronal migration. Nat. Neurosci. 7:1195-1203.
Tabata, H., and K. Nakajima. 2001. Efficient in utero gene transfer system to the developing mouse brain using electroporation: visualization of neuronal migration in the developing cortex. Neuroscience. 103:865-872.
Tabata, H., and K. Nakajima. 2003. Multipolar migration: the third mode of radial neuronal migration in the developing cerebral cortex. J. Neurosci. 23:9996-10001.
Tai, C. Y., D. L. Dujardin, N. E. Faulkner, and R. B. Vallee. 2002. Role of dynein, dynactin, and CLIP-170 interactions in LIS1 kinetochore function. J. Cell Biol. 156:959-968.
Tanaka, T., F. F. Serneo, C. Higgins, M. J. Gambello, A. Wynshaw-Boris, and J. G. Gleeson. 2004. Lis1 and doublecortin function with dynein to mediate coupling of the nucleus to the centrosome in neuronal migration. J. Cell Biol. 165:709-721.
Xiang, X., A. H. Osmani, S. A. Osmani, M. Xin, and N. R. Morris. 1995. NudF, a nuclear migration gene in Aspergillus nidulans, is similar to the human LIS-1 gene required for neuronal migration. Mol. Biol. Cell. 6:297-310.
Yan, W., A. H. Assadi, A. Wynshaw-Boris, G. Eichele, M. M. Matzuk, and G. D. Clark. 2003. Previously uncharacterized roles of platelet-activating factor acetylhydrolase 1b complex in mouse spermatogenesis. Proc. Natl. Acad. Sci. USA. 100:7189-7194.

What is claimed is:

1. An isolated nucleic acid that encodes an inhibitory RNA that inhibits Lissencephaly1 (LIS1) function, the nucleic acid comprising a nucleic acid sequence as listed in SEQ ID NO: 1, SEQ ID NO: 2, or SEQ ID NO: 5.

2. An isolated nucleic acid that encodes an inhibitory RNA that inhibits Dynein function, the nucleic acid comprising a nucleic sequence as listed in SEQ ID NO:6.

3. A method for treating a disease or disorder associated with abnormal growth or migration of neural progenitor cells in a subject, the method comprising administering to the subject an effective amount of a nucleic acid that encodes an inhibitory RNA that inhibits LIS1, dynein or dynactin function.

4. A method for treating a disease or disorder associated with abnormal growth or migration of neural progenitor cells, the method comprising administering to a subject an effective amount of a nucleic acid that encodes a dominant negative polypeptide that inhibits LIS1, dynein or dynactin function.

5. The method of claim 3, wherein the nucleic acid sequence is from about 15 to about 35 nucleotides in length.

6. The method of claim 3, wherein the nucleic acid sequence comprises a sense and anti-sense strand which form an RNA duplex.

7. The method of claim 3, wherein the sense and antisense strands are covalently linked by a single-stranded hairpin.

8. The method of claims 3, wherein the nucleic acid sequence comprises a non-nucleotide molecule.

9. The method of claim 3, wherein the nucleic acid sequence is at least about 95% identical to any one of SEQ ID NOS: 1, 2, 5, or 6.

10. The method of claim 9, wherein the nucleic acid comprises an addition, substitution, deletion or insertion of one or more nucleotides compared to any one of SEQ ID NOS: 1, 2, 5 or 6.

11. The method of claim 3 or 4, wherein the nucleic acid is comprised in an expression vector.

12. The method of claim 3 or 4, wherein the disease or disorder is a cancer of the central nervous system.

13. The method of claim 3 or 4, wherein the disease or disorder is glioma, meningioma, medulloblastoma, neuroectodermal tumor, epyndymoma, or any combination thereof.

14. The method of claim 3 or 4, wherein abnormal growth or migration is undesirable.

15. The method of claim 3 or 4, wherein the nucleic acid is administered to the disease or disorder site.

16. The method of claim 12, wherein the nucleic acid is administered to the cancer site.

17. The method of claim 3 or 4, wherein the nucleic acid is administered intracranially.

18. A method for identifying an agent that inhibits LIS1 function, wherein the method comprises:
a) contacting a cell with an agent,
b) determining whether the cell exhibits reduced LIS1 function, wherein reduced LIS1 function is indicative of an agent that inhibits the function of LIS1.

19. The method of claim 18, wherein the cell is neural progenitor cell, or cell of neural lineage, or a cell derived from a glioma.

20. The method of claim 18, wherein the determining step comprises comparing levels of cell proliferation, morphogenesis or cell migration by the cell in the presence of the agent with the levels determined in the absence of the agent.
